SEQUENCE LISTING (32 sequences)

SEQ ID NO: 1 - DNA, 5163 bp, Trichoderma reesei
  SEQ1 native gene
atggtttctggcgactacgccttcaaccccgatcaacacggcgcatatgccgaaccgtaccaacagccggacgacggccggactaggacgctgcttgacaaccaagccttcttttctgacttcgcgggccagcagcactacgaacagaaccagatgggcgactatggtggccctagatactccggcgatgccttctctccgacagcagccatggctcctccgatgctcactgccaacgacatgcctccacccgagatattggagtaccaggctccgctcgagccaagagaggtcccctttgccattcaggatccccacgacaacaacacggccatgtcttcgttcgacaacatggctgcggtactccgtcaccgtgcccgcaccactgccaaaagacctgcatattgggtcctggacagcaagggcaaggaggtggcatccattacatgggacaagctggcgtcaagagcggagaaagttgcacaagtcatccgagacaaaagtcctctttaccgtggcgatcgagttgccttgatctatcgtgatagcgaaatcattgacttcgccattgccttgctgggttgcttcattgctggagttgtggctgtcccgatcaatgacttgcaagactaccagcgcctcaactatattctcacctcgactcaggcgcatctggctcttactaccgaaaacaacctcaagaccttccagagagacattactgcgcagaagctcacgtggcctaaaggggtcgagtggtggaagaccaacgagttcggcggttaccatccgaagaagaaggaagacgcacctccgttaactgttcccgacctggcctatattgagttttcgcgagcaccaaccggcgacttgaggggcgttgttctcagccacaggacaatcatgcaccagatggcctgcctcagtgccataatctctaccgttcccaccaacggccccggcgataccttcaactcgacgttgcgggacaagaacggaaagctcatcggcggcggagccagcagcgagatattgctctcctatctggatccccgacagggcgtgggcatgattctcggcgttttgctgaccgtttacggcggccacactaccgtctggttcgatcacaaagccgtcgagtcgcctggcttatacgcgcatctgattaccaagtacagagcgacgattatgattgcggattaccccgggttgaagcgagctgcctacaactaccagcaagaccccatgacgacacgaaacttcaaaaaggggatggaacccaacttccaagcggtgaagctgtgcttgattgataccctgaccattgatagcgagttccatgaagttctggccgatagatggctgcggcccctgcgaaatccgcgagcgcgcgaggtcgtggcgccgatgctctgcctccccgagcatggcggcatgatcattagcgttcgagactggctcggcggtgaagaacgactgggagttccgctgaaactggacgagtctgacagggagtcggatgacgagaaagaagaggaagagaagccggccccgtcaaacggatttggtagcttgcttggtggtggagcagcgacaaccaaggagcaggacgagaagattgagttgggcgaggttatccttgaccgagaggctctcaagaccaacgaggttgtcgtcttggctcatggcaacgaagctaggaagaagacgtcgctggagcccaccatggtccgggtcggcgcctttggataccctatcccagatgccacgcttgctgttgtggaccctgagactggcctcctggcagcgccgcacacgattggcgagatctgggttgactctccgtctctctctggaggcttctgggcgcagccaaagaacaccgagctcatcttccacgcgcgtccgtacaagttcgagcctggcgagccgacgccaactgccgtggagccggaattcctgcgaaccggcctgcttggcacagtcatcgagggcaagatctatgtgctaggattgtacgaggatcggatacgacaaaaggtcgaatgggttgagcacggccacgcgggtatcgccgagtatcgctacttctttgttcagcacattgtggtgagcatcgtcaagaatgtccccaagatccacgactgctctgcctttgacgtctttgtcaatgacgagcacttgcctgtcgtggtcctcgagtctgccgcagcatcaacggcgcctctcacttcgggcggcccccctgtccagcctgacacggttctgttagactcgctggcggagaaatgcatggaggtgctcatgcaggagcaccatcttcgggtttactgcgttatgatcacagccccgaacgcactgccgcgagtgatcaagaacggaagacgggaaatagggaacatgctctgccggcgcgagtttgaccttggcaacctcccatgcgtgcatgtcaaatttggcgtcgaacatgcggttctcaacctcccgattggcgttgaccccattggtggtatctggtcaccaatcgcctcggactcgagaatcaatatcctggctcccgccgataagcagtattctggaatcgaccgcagagaggttgttatggacgaccggacgtctacaccgctcaacaatttcaagaccatcaccgatctgatccagtggcgtgttgctcgccagccagaggagctcgcttattgtaccattgacggcaggggcagagagggcaaggggattccgtggaagaagtttgactccaaggtggcggctgtggccatgtatctgaagaacaaagtcaaggtgcggccgggcgaccacctggtcctcatgtacacccactccgaggagtttgtctttgccgtccacgcgggaatcaaccttggcgcagtcattattcccatggcgccgcttgaccagaaccggctcaacgaagatgtccctgctttcctgcacctgatcgctgactacaaggttaaggcggtcctggtcaaccaggaagtggaccatttgctgaagctcaagatcgtgtcgagccacatcaaacagtccgcacagatcctgaagatctcgatgccgaatacctacaacacttcgaagccacctaagcagaacaacggtcttcgcgagcttgggctgacgatagatcccgcctggatcaggcctggataccccgtcctcatctggacatactggacgccggaccaacggagaatcgccgtccagctggggcatgataccatcctgggcatgtgcaaagtgcagaaggagacttgtcagatgacgagcttccagcccgttctcggttgcgtaagaagcacaacgggacttggtttcgtgcacacgtgcctgatgggcatctacgttggcaccgccacctacctgctgtctcctgtcgagttcgcccaaaatcccatctctctctttgttacgttgtcgaggtacaagatcaaggacacctatgcaacgccgcagatgcttgaccatgccatgtcgtcgatgcaggccaagggctttacaatgcacgaactgaagaatatgatgattactgcagagggccggccgcgggtagatgtattccagaaggtacggatgcattttgcgagcgccgggctggataggacggccatcaacacggtctactcgcatgtgctcaacccgatgattgcttcgaggtcttacatgtgcatcgagcctattgagctctggctcgacaccaaggctcttcgacgcggcctcgtcgtcccggtcgatcacgattcaaacccgcaagctcttctcctgcaggattccggcatggtgccggtgtctacccagattgccattgtcaaccccgagagccgcgcgcattgctacgatggagaatatggcgagatctgggtcgactccgaggcgtgcgtaaaggccttttacggctccaaggaagcgtttgacgtggagcgcttcgacggccggacggtcgacggcgaccccaacgtgcgatacgtgcgaactggtgacttgggctttttgtataatgtcaaccggcctatcgggcccaacggcgccctggtggagatgcaagtcttgtttgtgctcggtagcatcggcgagacttttgaaattaacggtttgagtcacttccccatggatattgagctgtcggtggaacgctgccaccgcaacattgtacccaacggctggtaagtacagggccaactcttctgtgagatgctacttgactaatagttggtgatgtgcagtgctgtattccaagctggtggcttggtcgtggtcctggtagaggtgagccgcaagtcttacctcgcctccatggtgccagtcattgtcaacgccatcttgagcgagcatcagatcgttgccgatattgtggcctttgtcaacaagggcgacttcccacgctctcgcctgggagagaagcagcgaggaaagatcctcgccggatgggttacgcgcaagatgcgcaccatggcccagttcgccatcagagatctcgacgccagcatgctcgagccgggtgagggtccggatgccaataggacctctacgggtagcctccgtagcctgggcgccgccgtcccgccaaacttcaagatggttggacaggcgccgcagatactggaacaggaggaatttacgcagcagatggatcacatggcccattcggagccggtcaggcatgccatggccgctccagaggagcagcaggcgccggcggcctattacgctggcggccaggaggctgctttcatgcagggctataatcagcagccgccaccaccaccaccaagcagccagggaggataccagtacgagcaattcgagccagtgcaagcacagcaacagtaccagccgcagtcacagcatcagtttgaaccatctcaagcctttgagccagcgcagcaatacgagccagcgcaacagcacgagcaggagccaaggccgatggactctcgaggacaagatgcgccgtccattgttgagccagagacctcagcctccgtgcctgatacgccgccgccgagaaacaggttgagtcaagggccgccccagatccggctcccgggcgttgacggacgggaagggctcgacttctggggaggcaacgacgagacggactggacggccgatgccatgatgcacatgaatctcactggcccacggtaa

SEQ ID NO: 2 - DNA, 2526 bp, Artificial Sequence
  Hygromycin B resistance marker
tgcaaggcgattaagttgggtaacgccagggttttcccagtcacgacgttgtaaaacgacggccagtgagcgcgacgtaatacgactcactatagggcgaattggcggaaggccgtcaaggcctaggcgcgccatgagctcgttaacaagacacagccctataacttcgtataatgtatgctatacgaagttatataacggtgagactagcggccggtccccttatcccagctgttccacgttggcctgcccctcagttagcgctcaactcaatgcccctcactggcgaggcgagggcaaggatggaggggcagcatcgcctgagttggagcaaagcggcccggccgccatgggagcagcgaaccaacggagggatgccgtgctttgtcgtggctgctgtggccaatccgggcccttggttggctcacagagcgttgctgtgagaccatgagctattattgctaggtacagtatagagagaggagagagagagagagagagagagaggggaaaaaaggtgaggttgaagtgagaaaaaaaaaaaaaaaaaaaaatccaaccactgacggctgccggctctgccacccccctccctccaccccagaccacctgcacactcagcgcgcagcatcacctaatcttggctcgccttcccgcagctcaggttgttttttttttctctctccctcgtcgaagccgcccttgttcccttatttatttccctctccatccttgtctgcctttggtccatctgcccctttgtctgcatctcttttgcacgcatcgccttatcgtcgtctcttttttcactcacgggagcttgacgaagacctgactcgtgagcctcacctgctgatttctctccccccctcccgaccggcttgacttttgtttctcctccagtaccttatcgcgaagccggaagaacctcttaacctctagatgaaaaagcctgaactcaccgccacgtctgtcgagaagttcctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatctcagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcacccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagatgcatggctttcgtgaccgggcttcaaacaatgatgtgcgatggtgtggttcccggttggcggagtctttgtctactttggttgtctgtcgcaggtcggtagaccgcaaatgagcaactgatggattgttgccagcgatactataattcacatggatggtctttgtcgatcagtagctagtgagagagagagaacatctatccacaatgtcgagtgtctattagacatactccgagaataaagtcaactgtgtctgtgatctaaagatcgattcggcagtcgagtagcgtataacaactccgagtaccagcgaaagcacgtcgtgacaggagcagggctttgccaactgcgcaaccttgcttgaatgaggatacacggggtgcaacatggctgtactgatccatcgcaaccaaaatttctgtttatagatcaagctggtagattccaattactccacctcttgcgcttctccatgacatgtaagtgcacgtggaaaccatacccaatataacttcgtataatgtatgctatacgaagttatagggctcttgtctgtt

SEQ ID NO: 3 - DNA, 1846 bp, Artificial sequence
  LIC reception vector
tttatcagggttattgtctcatgagcggatacatatttgaatgtatttagaaaaataaacaaataggggttccgcgcacatttccccgaaaagtgccacctgactaaaccagacagacagctgtctctcctctctaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagca

SEQ ID NO: 4 - DNA, 4436 bp, Artificial sequence
  pSEQ1flank5
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgactgaataacttcgggcggcccccctgtccagcctgacacggttctgttagactcgctggcggagaaatgcatggaggtgctcatgcaggagcaccatcttcgggtttactgcgttatgatcacagccccgaacgcactgccgcgagtgatcaagaacggaagacgggaaatagggaacatgctctgccggcgcgagtttgaccttggcaacctcccatgcgtgcatgtcaaatttggcgtcgaacatgcggttctcaacctcccgattggcgttgaccccattggtggtatctggtcaccaatcgcctcggactcgagaatcaatatcctggctcccgccgataagcagtattctggaatcgaccgcagagaggttgttatggacgaccggacgtctacaccgctcaacaatttcaagaccatcaccgatctgatccagtggcgtgttgctcgccagccagaggagctcgcttattgtaccattgacggcaggggcagagagggcaaggggattccgtggaagaagtttgactccaaggtggcggctgtggccatgtatctgaagaacaaagtcaaggtgcggccgggcgaccacctggtcctcatgtacacccactccgaggagtttgtctttgccgtccacgcgggaatcaaccttggcgcagtcattattcccatggcgccgcttgaccagaaccggctcaacgaagatgtccctgctttcctgcacctgatcgctgactacaaggttaaggcggtcctggtcaaccaggaagtggaccatttgctgaagctcaagatcgtgtcgagccacatcaaacagtccgcacagatcctgaagatctcgatgccgaatacctacaacacttcgaagccacctaagcagaacaacggtcttcgcgagcttgggctgacgatagatcccgcctggatcaggcctggataccccgtcctcatctggacatactggacgccggaccaacggagaatcgccgtccagctggggcatgataccatcctgggcatgtgcaaagtgcagaaggagacttgtcagatgacgagcttccagcccgttctcggttgcgtaagaagcacaacgggacttggtttcgtgcacacgtgcctgatgggcatctacgttggcaccgccacctacctgctgtctcctgtcgagttcgcccaaaatcccatctctctctttgttacgttgtcgaggtacaagatcaaggacacctatgcaacgccgcagatgcttgaccatgccatgtcgtcgatgcaggccaagggctttacaatgcacgaactgaagaatatgatgattactgcagagggccggccgcgggtagatgtattccagaaggtacggatgcattttgcgagcgccgggctggataggacggccatcaacacggtctactcgcatgtgctcaacccgatgattgcttcgaggtcttacatgtgcatcgagcctattgagctctggctcgacaccaaggctcttcgacgcggcctcgtcgtcccggtcgatcacgattcaaacccgcaagctcttctcctgcaggattccggcatggtgccggtgtctacccagattgccattgtcaaccccgagagccgcgcgcattgctacgatggagaatatggcgagatctgggtcgactccgaggcgtgcgtaaaggccttttacggctccaaggaagcgtttgacgtggagcgcttcgacggccggacggtcgacggcgaccccaacgtgcgatacgtgcgaactggtgacttgggctttttgtataatgtcaaccggcctatcgggcccaacggcgccctggtggagatgcaacttgtttgtgctcggtagcatcggcgagacttttgaaattaacggtttgagtcacttccccatggatattgagctgtcggtggaacgctgccaccgcaacattgtacccaacggctggtaagtacagggccaactcttctgtgagatgctacttgactaatagttggtgatgtgcagtgctgtattccaagctggtggcttggtcgtggtcctggtagaggtgataacaagacacagcccgggctcttgtctgttactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 5 - DNA, 4572 bp, Trichoderma reesei
  SEQ5 native gene
atgagggcctatcagatcgagatgctcgacaagagcctcaagcaaaatgtcattgttgctgtatgttgaagtttctctccaatcccccgtctccccctttgctgtcgttgtcttcgacgttgaaagacatgtccattgaccaaggggcgttgttataaatctagatggacacgggaagtggcaagactcaagtgtaagttgtgcatcttcatcatcggcagcccacgtaacctgtgccagcccttagcacccttcttcgcaaaagactgacttggcgcttgcatcagagctgtgcttcgtatcaagaaggagctggaaatctgcgatgcatcaaaggtgagtctgccgtctggatacagttgcacaacgacctggacagctgcactgacgcagcacgcatcagatcatctggttcatcgcgccaacagtttcgctgtgtcatcagcaacacgatgtgctcaagttgcagatacctgccgtgcccatgatgacactggccgggaactccaatatcgatgcttgggggccggatatctgggccattcttctcgacacggttcgaattgtcatatccacaccccaggttctgctcgatgcccttgaccatgcttacctgaacttgggtcttctggcgctgcttgtatttgatgaaggtatgggacgacctgccttcactctgtaaaggcaaaggggccgccagaagttgcaaatcgctgacgtgtcttgtgcaaaagtccacaactgcattggcagaagtccaggcggcaaaatcatgctccaccactaccatccgcgcaagctggctggtgaaagcgtgcctgctgttctgggtctgacggcaactccgagcattcagtctgagcttgccgatattgatgccttggaatggctgatggatgcaagatgcgtctcgcccactctccatcgcgacgaactgctcaaatgcgtcaagaggcccaatatcaagcacatcatctataaagccggcaaagaagacatcacgacgcccaccatgcgcgacttggatcgggtctaccgggcgctggacattctcgaagacccctacatactcatgctgcgcaacaaccctacggaccgaaacaaccgcctgctgctaacagccattgaaaagtacgatacctacacacagaaccagatgaagtcgttctgcgcccgatcaagagagatatgcaagcaactcggtccctgggctgctgacctcttcatctggaaggccatctcagctcacttggacaaggtggacaggcagacggatggagttgacgagtatggcaacaagtggtcgtcggggtcgacaagcttcctggaaaagaagcacctggccgacatctatcgtcgagtcaaggtccaacgtccttccgatgtgccacaggtctttgaagacatttccgacaaggtcggtaagctaatctttgagcttctgtcggtagaggagcccacggtgggcatcatcttcgtcgaggaacgagtcatggttgctatgctggccgaggttctctctgtcaaccacacaatcacgtcccggtaccggatcgggaccatggttggcacctcaaattacgctgggcggcggaaggccgtttatgacttcgaccagaaaacggactacaaggacctgcagagcttccgctccggcaagattaacctgctgattgcgacgtcagtgctggaggagggcatcgacgtgcctgcctgcaacctagtcatatgctttgacactccgacgaccccaaagtcctttatccagcggcgcggacgggctcgctccaaggactcgaatctccttcttttctttgacgatgccaaccctgcgatcttgaagtggcaggcgaaagaggaggagatgaacaggatcttcgaagacgaagagagggcgattcgcgaactcggcaaactggaagattcggagagtccgagcaccatctccttcaccgtcccgtctaccggcgcaaggctagattttgacaatgcgaagcagcacctcgagcacttctgcagagtcttgtgcccgtcggactttgtggacagccgcccggactacatcatccgcagggagcaggactctcctttgttgactgccattgtactgctccctccgtttctgccggtgaatctgaggcagcacaccagtgcttctccttggcgctccgagaagaacgccaccaaggatgctgcgtatcaggcgtatatagccctgtatgacgcgaagctcgtcaacgagaacctgctgcccttcaagtccagcgacatgctcggaatcgataagcgagtatccgaggtgccggtcgagccgttgatgaagccatggcatcgtgtcgctcctgcgtggcgggaagctggcgacaagtggctttactccttgagctgcgtggaggaggacggccgagtaagtgcagagtacgaggttctgctgccagtctggctgaaccagcctcagcccctgaaaatgttcctcgaccgcaatcaccaggtggagttgcagctgaaggccgggatacccgtgccgcacgagcaagttgcgtccctgccagatcatacatcgactttgctggcgctgcatttcggtcatcgatggcctctcgagcagaaagagcacgtcattcgggtctgggccaaggatcaacccctatcgctgaaccaaattggcgagctcacatacgatccacagaatgagagcgtcagccggggagagtttctcatccgggacaacaccagagccccctacctgtacaaggataccattgcgttcaagcccgaaccgagccaggtccagaataccttttacgagtacgacaaggcgcccgaagacgtgccgtatctcgtgctcaccaaatggacgcggcggaccgactttctgcatcgcctccaagggaatcccgccaagaatgaggttagtagcaagccatacgcacgcgtatatccgctgtcgtgggcgacagtcgataccatccccgccaggcacgcccagtttggcatgctgatcccgaccatgatccacgagctcggcgtcatgctcatggccaaggagctggcctactccgttctcgacgaggttggcatttcggatctgcagctggtcaaggaggccatcagcgcgcggagtgcctcggagccggtgaattacgagaggctggagtttttgggcgactcgattctcaagttttgtgcctgtatgcgcgccgctgctgaaagtaagttgctcaagcgttttactcatatatgactcctgtgtgcacctgtcctctgacatggaactgttttgctgaccacatttgatactgcctagaacccgactatcccgagggctatctctcgtattggagagaccgactcgtctccaactcgaggctgtacaaagccgctctcgagtttgggctgccgaggttcatcttgacgaaaccttttaccggtcaaaagtggcgcccactctacctggacgaggtcctccagcaaggggacgtcgctacgccggagaagagaaaattatcgaccaagacgctcgcagacgtggtcgaggcgctgatcggggcctcatacgtcgatggaggcctttcaaaggcagtgacttgcatctcaaaattcgtccccgaaggctcgtggaccagtgttgatgcagatagagagtctctctttgcgagagtgccagacggcgagcctctcccgccgccattggagccgctggagaagttgatcggctacacgttccagaaaaaggcgctcttgatggaggctctgacgcatgcctcgtatgctgcagacttcggaacgcgatctctcgagaggctcgaattcataggagacgctgtcctggacaacattatcgttacgaagctctttaggctgaagccagcgctgccccatttcaggatgcatacgctgaagacgggcctggtgaatggggactttcttgctttcatgacaatggagcacggagtgcaactggcggcggaccctgtggtgacagaagaagctacggtggaggtcccggaaacgatttcctacctgtggtcgtttttgaggcaggcctcttttcccattgccatcgagctgaaggagacgaacaagcggcacgctgccctgagagagcagattcacgaagcaatggacaatgacgatcattacccctgggcgctgctggccgccctgagcccgaagaagttctactctgacctcttcgaggcggttctcggcgctgtgtggatcgactccgggtcgctggcggcgtgcgagggcatggttgcgcagtttgggatcttaaagtacatggatcggctgctgcgtgacgaagtccacgtgcagcatcctaaggaggagctgggcatgtgggcaaacacagagactgtgacgtacgagctcgagatgaaggggagcgaggagagcgcgggggagagggagtatttctgcaaggtgtttgttggaaagagggaggttgtggaggttcgtgggggggtcaataaggaggaggtgaagacgaagggtgcgacggaggcgttgcggattttgagggaggagaaaaggcgcggtgctgaggatgtggtgatggtgggataa

SEQ ID NO: 6 - DNA, 20 bp, Artificial sequence
  hygrfw
tgcaaggcgattaagttggg

SEQ ID NO: 7 - DNA, 54 bp, Artificial sequence
  hygrrv
cggcgaggatctttcctcgctgcttctctcaacagacaagagccctataacttc

SEQ ID NO: 8 - DNA, 20 bp, Artificial sequence
  SEQ1fl3fw
ttgtcaacgccatcttgagc

SEQ ID NO: 9 - DNA, 20 bp, Artificial sequence
  SEQ1fl3rv
accaaccagtccatcctctg

SEQ ID NO: 10 - DNA, 36 bp, Artificial sequence
  fus1
aaaccagacagacagtatacgactcactatagggcg

SEQ ID NO: 11 - DNA, 50 bp, Artificial sequence
  fus2
gttaacagacaagagcccgaagttattcgggtagtagagtttgaaagggg

SEQ ID NO: 12 - DNA, 38 bp, Artificial sequence
  fus3
agagaggagagacagtgttaacagacaagagcccgaag

SEQ ID NO: 13 - DNA, 20 bp, Artificial sequence
  SEQ1MKOfw
atgtgctaggattgtacgag

SEQ ID NO: 14 - DNA, 20 bp, Artificial sequence
  SEQ1MKO1rv
ataatagctcatggtctcac

SEQ ID NO: 15 - DNA, 20 bp, Artificial sequence
  SEQ1MKO2rv
ttgacaaaggccacaatatc

SEQ ID NO: 16 - DNA, 20 bp, Artificial sequence
  M1Seq-01
atcgctacttctttgttcag

SEQ ID NO: 17 - DNA, 20 bp, Artificial sequence
  M1Seq-02
cagcttggaatacagcactg

SEQ ID NO: 18 - DNA, 20 bp, Artificial sequence
  SEQ5M5fw
gactctctatctgcatcaac

SEQ ID NO: 19 - DNA, 54 bp, Artificial sequence
  SEQ5M5rv
tgacctggaaagctttcaatgtagaggtagactagtcaaagaagacatcacgac

SEQ ID NO: 20 - DNA, 54 bp, Artificial sequence
  SEQ5M3fw
cgcatggtgggcgtcgtgatgtcttctttgactagtctacctctacattgaaag

SEQ ID NO: 21 - DNA, 20 bp, Artificial sequence
  SEQ5M3rv
gattacctgtcaagtctatg

SEQ ID NO: 22 - DNA, 32 bp, Artificial sequence
  SEQ5Mnestfw
gacagtcctgcaggagtcactgcctttgaaag

SEQ ID NO: 23 - DNA, 33 bp, Artificial sequence
  SEQ5Mnestrv
gacagtcctgcaggtgtaaggataaaggacgac

SEQ ID NO: 24 - DNA, 39 bp, Artificial sequence
  LIC1fw
ctaggagttctgccttgggtttaaacgagagaaagactc

SEQ ID NO: 25 - DNA, 39 bp, Artificial sequence
  LIC1rv
ctaggagtctttctctcgtttaaacccaaggcagaactc

SEQ ID NO: 26 - DNA, 3414 bp, Emericella nidulans
  amdS
ggatgtacgacgtatatccatctttaactagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactgcattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatattggctgggttgatggctgcttcgagtgcagtctcatgctgccacaggtgactctggatggccccataccactcaacccatggtacccgtgcctcaggggtgagctggttgttgccttgcggtagagtaataacgatagctcagccttgcaggtgatttccgcgtctgtctattgtccttattactgtgtcgagtccccaagttttcttccaatagacatca

SEQ ID NO: 27 - DNA, 36 bp, Artificial sequence
  SEQ5MamdSfw
gttctgccttgggtttaggatgtacgacgtatatcc

SEQ ID NO: 28 - DNA, 41 bp, Artificial sequence
  SEQ5MamdSrv
gtctttctctcgtttatgatgtctattggaagaaaacttgg

SEQ ID NO: 29 - DNA, 19 bp, Artificial sequence
  SEQ5MKO1fw
actctctatctgcatcaac

SEQ ID NO: 30 - DNA, 18 bp, Artificial sequence
  SEQ5MKO1rv
gatccccgatttctttgg

SEQ ID NO: 31 - DNA, 20 bp, Artificial sequence
  SEQ5MKO2fw
tgatgtgcttgatattgggc

SEQ ID NO: 32 - DNA, 20 bp, Artificial sequence
  SEQ5MKO2rv
ctccatcgctcaactatgtg

(19) United States
(12) Patent Application Publication
(10) Pub. No.: US 2023/0416713 A1
(43) Pub. Date: Dec. 28, 2023

(54) Process for the production of a technical enzyme composition with low viscosity produced by a filamentous fungus

(71) Applicant: Clariant Produkte (Deutschland) GmbH, Frankfurt am Main (DE)

(72) Inventors: Christian GAMAUF, München (DE); Jörg CLAREN, Gauting (DE)

(21) Appl. No.: 18/036,805

(22) PCT Filed: Nov. 12, 2021
(86) PCT No.: PCT/EP2021/081510

§ 371 Date: May 12, 2023

(30) Foreign Application Priority Data
Nov. 12, 2020 (EP) 20207123.9

(51) Int. Cl. C12N 9/24

(52) U.S. Cl. CPC C12N 9/2402; C12Y 302/01004

(57) ABSTRACT

The present invention relates to a process for the production of a technical enzyme composition with low viscosity produced by a genetically modified filamentous fungus, a genetically modified filamentous fungus suitable for production of the technical enzyme composition, the use of such a genetically modified filamentous fungus for the production of the technical enzyme composition with low viscosity and a technical enzyme composition with low viscosity produced by such a process.

The present invention relates to a process for the production of a technical enzyme composition with low viscosity produced by a genetically modified filamentous fungus cell, a genetically modified filamentous fungus cell suitable for production of the technical enzyme composition, the use of such a genetically modified filamentous fungus cell for the production of the technical enzyme composition with low viscosity and a technical enzyme composition with low viscosity produced by such a process.

Enzymes are important components of many commercial products and respective production processes. Modern laundry compositions contain a wide variety of different enzymes such as cellulases, many feed products for livestock contain enzymes and enzymes are also used for the production of many commercial products such as the production of bioethanol, of plastic alternatives/biodegradable plastics or even food products. Enzymes used in such processes are often called “industrial enzymes” or “technical enzymes”.

To attain economic feasibility of the desired end product, a high yield and low production cost of the used technical enzyme(s) is a necessity. This applies in particular when the desired commercial end product is a bulk product which has to compete with low price alternatives originating from cheap mineral-oil derived chemical synthesis processes.

Filamentous fungi are well known as effective producers of a wide variety of technically feasible enzymes. In addition, filamentous fungi are able to grow on a diverse range of substrates.

However, the implementation of filamentous fungi for the production of technical enzymes is still not very popular as the high viscosity of the fermentation broth of such fungi often affords time and cost consuming measures leading to too high production costs of the technical enzyme composition. In order to obtain a high yield of enzymes, a strong growth of the fungus is desired, however, strong growth results in a high content of fungus biomass within the fermentation broth. Fungi, which are known to consist of i.a. hyphae are known within the art as rendering any fermentation substrate into a high-viscous composition. This effect is significantly more distinct when a filamentous fungus is used which exhibits a sponge-like, slimy appearance.

High viscosity causes many problems, as the fungus needs constant oxygen supply by aeration during growth. In addition, cooling of the fermenter, especially in industrial-scale production is required. Both can only be guaranteed by constant stirring—on the one hand to distribute the air bubbles homogenously within the broth, and on the other hand to facilitate constant heat-exchange with the cooling devices. The higher the viscosity of the broth the more energy needs to be spent to realize effective stirring within the reactor. Further, more air has to be pressed into the reactor causing also higher energy consumption within the compressor and sterile-filter unit. Thus, both CAPEX and OPEX increase with increasing viscosity of the fermentation broth. An alternative measure—less cell mass production—is also not attractive for commercial production as this would always be accompanied by a lower yield of technical enzyme production.

The inventors of the present invention have therefore set themselves the task to develop a process for the production of a technical enzyme composition with low viscosity produced by a filamentous fungus while maintaining a high yield of enzymes.

The task has been solved by a process for production of a technical enzyme composition, comprising the following steps:
(a) providing a fermentation medium with a glucose content of from 5 to 550 g/L;
(b) addition of at least one filamentous fungus cell wherein SEQ ID NO:1 has been disrupted;
(c) mixing of the fermentation medium and the at least one filamentous fungus cell for a time period of from 1 minute to 10 days at a temperature of from 20 to 35° C.;
(d) obtaining a technical enzyme composition.

It is of particular advantage of the inventive process that a high yield of target enzymes is achieved with any kind of medium which contains a significant amount of glucose. The majority of the proteins secreted by filamentous fungi are enzymes that degrade naturally occurring polymers such as cellulose and hemicellulose and the availability of glucose would usually prevent the filamentous fungus from producing such enzymes as they are not needed for metabolization of glucose. Further, no addition of expensive inducing substances such as gluco-oligosaccharides or sophorose is necessary. Therefore, a wide variety of different fermentation substrates which are readily and cheaply available may be used.

Within the present invention the term “technical enzyme composition” is to be understood to consist of or to contain a partly or completely fermented medium and may even contain components of the original medium but also any compound generated during the fermentation process such as enzymes. A “technical enzyme composition” may also contain part of or all of the microbial biomass of the fermentation microorganism i.e. the filamentous fungus.

Within the present invention the technical enzyme composition preferably contains at least one enzyme belonging to the class of hydrolases and/or at least one enzyme belonging to the class of oxidoreductases. Within a particularly preferred embodiment of the present invention, the technical enzyme composition contains at least one enzyme belonging to the class of hydrolases and/or at least one enzyme belonging to the class of oxidoreductases which has been produced by the at least one filamentous fungus cell. Within another also particularly preferred embodiment, the technical enzyme composition contains at least one enzyme belonging to the class of cellulases and/or at least one enzyme belonging to the class of hemicellulases which has been produced by the at least one filamentous fungus cell.

Within the present invention, the term “enzyme belonging to the class of hydrolases” is to be understood as comprising any enzyme, capable of the hydrolysis of a chemical bond. Enzymes belonging to the class of hydrolases are classified as EC 3 in the EC number classification of enzymes. According to the present invention, the term “hydrolases” comprises cellulases, hemicellulases and may also encompass pectinases, oxidases, chitinases, chitosanases, transglutaminases, pentosanases, niringinases, limoninases, lactonases, nucleases, ureases, lipoxygenases, esterases, alpha-glucanases, phosphatases, isomerases, proteases and accessory proteins.

Within the present invention, the “enzyme belonging to the class of hydrolases” may be a native enzyme of the filamentous fungus or a heterologous enzyme originating from a different species of microorganism, in particular from a different species of filamentous fungus but may also originate from a non-filamentous fungus or a bacterium.

As used within the present invention, the term “cellulase” refers to any enzyme capable of hydrolyzing cellulose polymers to shorter oligomers and/or glucose. Cellulases preferred within the technical enzyme composition include cellobiohydrolases (CBH) (EC 3.2.1.-), endo-1,4-β-glucanases (EG) (EC 3.2.1.4).), beta-glucosidase (EC 3.2.1.4), cellobiose hydrolase (EC 3.2.1.21), glycoside hydrolase 61 (GH61 and CBM33).

As used within the present invention, the term “hemicellulase” refers to any enzyme capable of degrading or supporting the degradation of hemicellulose. Hemicellulases preferred within the technical enzyme composition include ρ-glucanases (EC 3.2.1.-), endo-xylanases (EC 3.2.1.8), ρ-xylosidases (EC 3.2.1.37), acetylxylan esterase (EC 3.1.1.72), acetylgalactan esterase (3.1.1.6), acetyl mannan esterase, feruloyl esterase (EC 3.1.1.73), glucuronoyl esterase (EC 3.1.1.-), α-L-arabinofuranosidase (EC 3.2.1.55), α-arabinopyranosidase (3.2.1.-), α-galactosidase (EC 3.2.1.22), ß-galactosidase (EC 3.2.1.23), α-glucuronidases (EC 3.2.1.139), β-mannase (EC 3.2.1.78), β-mannosidases (EC 3.2.1.25), mannan 1,4-mannobiosidase (EC 3.2.1.100), arabinogalactan endo-beta-1,4-galactanase (EC 3.2.1.89), endo-beta-1,3-galactanase (EC 3.2.1.90), galactan endo-beta-1,3-galactanase (EC 3.2.1.181, glucuronoarabinoxylan endo-1,4-beta-xylanase (EC 3.2.1.136), alpha-L-fucosidase (EC 3.2.1.51), coniferin beta-glucosidase (EC 3.2.1.126), xyloglucan hydrolases (EC 3.2.1.150, 151, 155), xylan α-1,2-glucuronosidase (EC 3.2.1.131), endo-xylogalacturonan hydrolase (EC 3.2.1.-; GH28), α-amylase (EC 3.2.1.1), glucan 1,4-α-glucosidase (EC 3.2.1.3), galactan 1,3-galactosidase (GH43), -1,4,-endogalactanase (EC 3.5.1.89; GH53), a-rhamnosidase (EC 3.2.1.40) and ß-rhamnosidase (EC 3.2.1.43).

As used within the present invention, the term “pectinase” refers to any enzyme capable of degrading or supporting the degradation of pectin. Pectinases preferred within the technical enzyme composition include polygalacturonases (EC 3.2.1.15, 67, 82; GH28 pectin methyl esterase (EC 3.1.1.11), pectin acetyl esterase (EC 3.1.1.-), rhamnogalacturonase (EC 3.2.1.-; GH28), rhamnogalacturonan acetylesterase (EC 3.1.1.86), rhamnogalacturonan galacturonohydrolase (EC 3.2.1.-), xylogalacturonan hydrolase (EC 3.2.1.-), pectin methylesterase (EC 3.1.1.11), beta-arabinofuranosidase (EC 3.2.1.55), beta-1,4-galactanase (EC 3.2.1.89), beta-1,3-galactanase (EC 3.2.1.90), beta-galactosidase (EC 3.2.1.23), alpha-galactosidase (EC 3.2.1.22), feruloyl acetyl esterase (EC 3.1.1.-), alpha-fucosidase (EC 3.2.1.51), (beta-fucosidase) (EC 3.2.1.38), beta-apiosidase (EC 3.2.1.-), alpha-rhamnosidase (EC 3.2.1.40), beta-rhamnosidase (EC 3.2.1.43), alpha-arabinopyranosidase (EC 3.2.1.-), beta-glucuronidase (EC 3.2.1.31), alpha-glucuronidase (EC 3.2.1.139), beta-xylosidase (EC 3.2.1.37) and alpha-xylosidase (EC 3.2.1.x).

As used within the present invention the term “accessory protein” refers to any enzyme capable of supporting cellulolytic enzyme activity. The term is well known to a person skilled in the art. Preferred accessory proteins within the technical enzyme composition include Expansin, Swollenin, Loosenin and CIP Proteins (EC 3.1.1.-; CE15).

As used within the present invention, the term “oxidoreductase” refers to any enzyme capable of catalyzing an oxidation and/or a reduction reaction. Enzymes belonging to the class of oxidoreductases are classified as EC 1 in the EC number classification of enzymes. Oxidoreductase enzymes preferred within the technical enzyme composition include lytic polysaccharide monooxygenase (LPMO) (AA9-11; previously GH61 and CBM33, resp.) (EC 1.14.99.53-56, 1.14.99.610), lignin peroxidase (EC 1.11.1.14), manganese peroxidase (EC 1.11.1.13), aryl-alcohol oxidase (EC 1.1.3.7), glyoxal oxidase (EC 1.1.3.), carbohydrate oxidases (EC 1.1.3.4, 9, 10), cellobiose dehydrogenase (EC 1.1.99.18), catalase (hydrogen-peroxide oxidoreductase) (EC 1.11.1.6 or EC 1.11.1.21), dye-decolorizing peroxidase (EC 1.11.1.19), laccase (EC 1.10.3.2), peroxidase (EC 1.11.1.x) and versatile peroxidase (EC 1.11.1.16).

As used within the present invention, the term “esterases” refers to any enzyme capable of cleaving an ester bond. Esterases preferred within the technical enzyme composition include acetyl esterases, glucuronoyl esterases, feruoyl esterases, lipases, cutinases and phospholipases.

As used within the present invention, the term “alpha-glucanases” refers to any enzyme capable of degrading alpha-linked oligo- and polysaccharides. Alpha-glucanases preferred within the technical enzyme composition include alpha-amylases, glucoamylases, pullulanases, dextranases, trehalases, lactases, invertases and maltases.

As used within the present invention, the term “phosphatase” refers to any enzyme capable of cleaving phosphoester bonds. Phosphatases preferred within the technical enzyme composition include phytases.

As used within the present invention, the term “isomerases” refers to any enzyme capable of transferring a chemical compound into an isomeric structure. Isomerases preferred within the technical enzyme composition include xylose isomerases, glucose isomerases and arabinose isomerases.

As used within the present invention, the term “proteases” refers to any enzyme capable of cleaving a peptide bond. Proteases preferred within the technical enzyme composition include serine proteases, threonine proteases, aspartic proteases, cysteine proteases, glutamic proteases and metalloproteases.

The enzymes referenced within the present invention are classified according nomenclatures that are either based on the International Union of Biochemistry and Molecular Biology's Enzyme Nomenclature and Classification (http://www.chem.qmul.ac.ukhubmb/enzyme/) or on Carbohydrate-Active EnZYmes (http://www.cazy.org/) database.

According to the present invention the term “fermentation medium” is to be understood as referring to any fermentation medium known to a person skilled in the art as suitable for the inventive process. Within the process of the present invention, the fermentation medium contains from 5 to 550 g/L glucose, wherein glucose contents from 5 to 450 g/L glucose, 5 to 420 g/L, from 8 to 400 g/L and from 10 to 280 g/L are preferred. Further preferred ranges of glucose are from 10 to 450 g/L, from 40 to 400 g/L and from 50 to 350 g/L. Also preferred ranges of glucose are from to 50 g/L, from 6 to 40 g/L or from 7 to 35 g/L and from 50 to 450 g/L, from 80 to 400 g/L and from 100 to 380 g/L. The glucose contained in the fermentation medium may originate from any source known to a person skilled in the art as suitable for the inventive process. Within a preferred embodiment, the glucose originates from corn, sugar cane or sugar beets, preferred sources are corn syrup, sugar cane or sugar beet molasses and mixtures thereof.

Within a preferred embodiment of the present invention the “fermentation medium” can at least partly originate from chemical, mechanical and/or enzymatic hydrolysis of lignocellulosic biomass and preferably comprises prior mechanical and/or acidic pretreatment of the lignocellulosic biomass. The fermentation medium originating from chemical, mechanical and/or enzymatic hydrolysis of lignocellulosic biomass may be used “as it is” or additional glucose can been added to the fermentation medium to obtain a desired total glucose content of the fermentation medium of from to 550 g/L. Glucose contents from 5 to 450 g/L glucose, 5 to 420 g/L, from 8 to 400 g/L and from 10 to 280 g/L are also suitable for the inventive process. Further preferred ranges of glucose are from 10 to 450 g/L, from 40 to 400 g/L and from 50 to 350 g/L. Also preferred ranges of glucose are from 5 to 50 g/L, from 6 to 40 g/L or from 7 to 35 g/L and from 50 to 450 g/L, from 80 to 400 g/L and from 100 to 380 g/L.

The hydrolysis of the lignocellulosic biomass has been carried out by mechanical and enzymatical hydrolysis or by sole enzymatic hydrolysis without the addition of any organic and/or inorganic acid(s). The hydrolysis of lignocellulosic biomass is known to a person skilled in the art, exemplary methods are for example described within Vishnu et al. 2012 (Trends in bioconversion of lignocellulose: Biofuels, platform chemicals & biorefinery concept in bioconversion of lignocellulose: Biofuels, platform chemicals & biorefinery concept. Progress in Energy and Combustion Science, August 2012, vol. 38 (4), 522-550) and Prasad et al. 2019 (Bioethanol production from waste lignocelluloses: A review on microbial degradation potential Chemosphere Volume 231, September 2019, p. 588-60).

Within the present invention the term “lignocellulosic biomass” is to be understood to comprise all kind of biomass known to a person skilled in the art as comprising lignocellulose. Particularly preferred lignocellulosic biomass according to the present invention includes wood, cereal straw such as but not limited to wheat straw, rice straw, barley stray, rye straw and oat straw, and/or husks and/or brans thereof, bagasse, oat hulls, switch grass, cellulose, raw paper pulp (obtained from pulp and paper production) and mixtures thereof. Additional components may comprise one or more of the following components: purified cellulose, pulp, milk whey or molasses. Lignocellulosic biomass which is particularly suitable for hydrolysis according to the process of the present invention is selected from the group consisting of cereal straw, cereal bran, cereal husks, wood, bagasse and mixtures thereof.

In a preferred embodiment the lignocellulosic biomass contains at least 25 wt.-%, preferably at least 40 wt.-%, more preferably at least 70 wt.-%, even more preferably at least 80 wt.-% and most preferred at least 90 wt.-% lignocellulose. It is to be understood that the lignocellulosic biomass may also comprise other compounds such as proteinaceous material, starch, sugars, such as fermentable sugars and/or non-fermentable sugars.

The fermentation medium originating from hydrolysis of lignocellulosic biomass has a high density of from 0.90 to 2.00 kg/L, preferably of from 0.95 to 1.90 kg/L, further preferred of from 1.00 to 1.50 kg/L and most preferred of from 1.05 to 1.35 kg/L.

The fermentation medium originating from hydrolysis of lignocellulosic biomass has a dry matter content of from 10 to 75 wt.-%, preferably of from 10 to 70 wt.-%, further preferred of from 20 to 65 wt.-%, from 30 to 65 wt.-% or from 40 to 60 wt.-% whereas a dry matter content of from 10 to 20 wt.-% and from 10 to 15 wt.-% is also preferred.

Within a preferred embodiment of the present invention, the fermentation medium further contains xylose and wherein the glucose to xylose ratio is selected from the range of from 1 to 3.5, such as a ratio selected from the range of from 1 to 3, from 1 to 2.8, of from 1 to 2.5 or of from 1 to 2.2. Further preferred ratios are 2.1, 2.0, 1.9 and 1.8.

Within an alternative preferred embodiment of the present invention, the fermentation medium further contains lactose and wherein the glucose to lactose ratio is selected from the range of from 1 to 10, such as a ratio selected from the range of from 1 to 9, from 1 to 8.5, of from 1 to 8 or of from 1 to 7. Further preferred ratios are 3, 4, 5 and 6.

Within a preferred embodiment of the present invention no gluco-oligosaccharides have been added to the fermentation medium and it is particularly preferred that the fermentation medium is free from gluco-oligosaccharides.

Within a preferred embodiment of the present invention no sophorose has been added to the fermentation medium and it is particularly preferred that the fermentation medium is free from sophorose.

Within another preferred embodiment of the present invention the fermentation medium contains less than 100 g/L cellulose and/or hemicellulose, preferably less than 80 g/L, more preferred less than 70 g/L, even more preferred less than 60 g/L, particularly preferred less than 50 g/L, and most preferred less than 40 g/L cellulose and/or hemicellulose. Within another preferred embodiment the fermentation medium of the present invention is free from hemicellulose. Within a further preferred embodiment of the present invention the cellulose content of the fermentation medium is selected from the range of from 0.01 g/L to 50 g/L, preferably from 0.1 to g/L, further preferred of from 1 to 30 g/L and most preferred of from 1 to 20 g/L.

Within another preferred embodiment the fermentation medium has a nitrogen content of from 0.05 to 2.0 g/L. Preferred contents of nitrogen are selected from the range of from 0.1 to 1.5 g/L, from 0.3 to 1.2 g/I or from 0.5 to 1.0 g/L. The nitrogen can be added in any form known to a person skilled in the art as suitable for the inventive purpose and may be added in form of ammonium sulfate, ammonia, urea, extracts from soy beans or combinations thereof. The amount of nitrogen can be added by feeding or by adding the total amount to the fermentation medium at any time before or during step (a) and/or (b) of the inventive process. It is thereby preferred that the nitrogen is added as a 25% (wt.-/wt.) solution of ammonia or a 40% (wt./wt.) solution of urea.

Within another preferred embodiment of the present invention, the fermentation medium contains from 0.5 to 80 wt.-% molasses, corn syrup or mixtures thereof, preferably from 5 to 75 wt.-%, from 15 to 70 wt.-%, from 25 to 65 wt.-%, from 35 to 60 wt.-% from 38 to 55 wt.-% or from 40 to 52 wt.-%.

Within a preferred embodiment of the inventive process the pH of the fermentation medium has been adjusted to a pH selected from the range of from pH 2.0 to pH 6.0, wherein ranges of from pH 3.0 to 5.5 and from pH 3.5 to 5.5 as well as from pH 3.5 to are particularly preferred. The adjusting of the pH can be carried out by any means and method known to a person skilled in the art as suitable for the inventive purpose. Within the process of the present invention the pH is preferably adjusted by addition of an acid such as sulfuric acid or acetic acid, NaOH, H₃PO₄ or ammonia.

Within a preferred embodiment of the inventive process the fermentation medium has a potassium hydrogen phosphate content of from 0.5 to 10.0 g/L, a magnesium sulfate heptahydrate content of from 0.05 to 1 g/L, a calcium chloride dihydrate content of from 0.1 to 1 g/L, an ammonium sulfate content of from 1.5 to 4.5 g/L, an iron (II) sulfate heptahydrate content of from 0.005 to 0.1 g/L, a manganese sulfate content of from 0.00001 to 0.001 g/L, a zinc sulfate heptahydrate content of from to 0.01 g/L and/or a copper sulfate pentahydrate content of from 0.0001 to g/L. Further preferred ranges are potassium hydrogen phosphate content of from 1 to 8.0 g/L, a magnesium sulfate heptahydrate content of from 0.1 to 0.8 g/L, a calcium chloride dihydrate content of from 0.3 to 0.8 g/L, an ammonium sulfate content of from 1.7 to 4.0 g/L, an iron (II) sulfate heptahydrate content of from 0.01 to g/L, a manganese sulfate content of from 0.0001 to 0.0008 g/L, a zinc sulfate heptahydrate content of from 0.002 to 0.008 g/L and/or a copper sulfate pentahydrate content of from 0.0002 to 0.008 g/L.

The “providing” of the fermentation medium according to step (a) of the inventive process can be carried out by any method and within any means known to a person skilled in the art as suitable for the inventive process. Within a preferred embodiment the fermentation medium is provided within a batch or fed batch reactor which is preferred equipped with a stirring device and a cooling device.

According to step (b) of the inventive process, at least one filamentous fungus cell wherein SEQ ID NO: 1 has been disrupted is added to the fermentation medium. Within another embodiment of the present invention at least one filamentous fungus cell wherein SEQ ID NO: 1 and SEQ ID NO: 5 have been disrupted is added to the fermentation medium. The addition of the at least one filamentous fungus cell can be carried out by any means and measure known to a person skilled in the art as suitable for the inventive process. Within a preferred embodiment, the at least one filamentous fungus cell is added in a quantity of from 10² to 10¹⁰ cells, preferably in a quantity of from 10³ to 10⁸ cells and most preferred in a quantity of from 10⁴ to 10⁷ cells per g of fermentation medium. The at least one filamentous fungus cell can thereby be added in dried form, as conidia or in form of a preculture, containing rest of preculturing medium. It is also possible to add the at least one filamentous fungus cell in form of a fully cultured medium (also referred to as main culture).

Within the present invention the term “filamentous fungus cell” is to be understood as any cell from any filamentous fungus existing in nature and/or known to a person skilled in the art. The term also comprises any filamentous fungus cell either of natural origin or modified. The term “modified” refers to genetically and non-genetically modified fungi. i.e. fungi which have been modified by genetic methods (e.g. transformation) and non-genetic methods e.g. chemical mutagenesis or irradiation, both of which are known to those skilled in the art. Within a preferred embodiment the at least one filamentous fungus cell is selected from the group consisting of Acremonium, Aspergillus, Chaetomium, Emericella, Fusarium, Humicola, Hypocrea, Irpex, Magnaporte, Myceliophthora, Neurospora, Penicillium, Rhizopus, Talaromyces, Trichoderma and Trametes, wherein Trichoderma and Aspergillus are particularly preferred, most preferred is Trichoderma reesei (teleomorph: Hypocrea jecornia).

Within another preferred embodiment of the present invention, the at least one filamentous fungus cell is a genetically modified filamentous fungus cell with the ability to express at least one heterologous hydrolyase or oxidoreductase enzyme, such as but not limited to an enzyme belonging to the class of cellulases, belonging to the class of beta-glucosidases or belonging to the class of xylanases or belonging to the class of lytic polysaccharide monooxygenases. Within such a preferred embodiment, the at least one heterologous hydrolase or oxidoreductase enzyme preferably originates from another filamentous fungus such as—but not limited to—Acremonium, Aspergillus, Chaetomium, Emericella, Fusarium, Humicola, Hypocrea, Irpex, Magnaporte, Myceliophthora, Neurospora, Penicillium, Rhizopus, Talaromyces, Trichoderma and Trametes. Within a particularly preferred embodiment the at least one filamentous fungus cell is a Trichoderma reesei cell and the at least one heterologous hydrolase or oxidoreductase enzyme originates from Acremonium, Ajellomyces, Alternaria, Armillaria, Arthroderma, Aspergillus, Bionectria, Bipolaris, Ceriporiopsis, Chaetomium, Cladophialophora, Clohesyomyces, Colletotrichum, Coniochaeta, Coniosporium, Diaporthe, Dothistroma, Emericella, Epicoccum, Exophiala, Fomes, Fonsecaea, Fusarium, Gibberella, Grosmannia, Hebeloma, Hortaea, Humicola, Hypocrea, Hypoxylon, Irpex, Isaria, Kuraishia, Leucoagaricus, Madurella, Magnaporthe, Marssonina, Metarhizium, Moniliophthora, Myceliophthora, Mycosphaerella, Neurospora, Oidiodendron, Ophiostoma, Paecilomyces, Paraphaeosphaeria, Penicillium, Phanerochaete, Phialophora, Pleurotus, Pochonia, Pseudocercospora, Pseudogymnoascus, Pyrenophora, Rasamsonia, Rhinocladiella, Rhizopus, Rhizosphaera, Rhynchosporium, Setosphaeria, Sphaerulina, Sporothrix, Stachybotrys, Stemphylium, Talaromyces, Termitomyces, Tilletiaria, Torrubiella, Trametes, Trichoderma, Trichophyton, Uncinocarpus and/or Valsa species.

According to the present invention, the at least one filamentous fungus cell as is a filamentous fungus cell wherein SEQ ID NO: 1 has been disrupted. The “disruption” can thereby be carried out by any means and measure known to the person skilled in the art as suitable for the purpose of disruption. The term “disruption” comprises all techniques that either lead to the gene no longer being transcribed or to the protein encoded by the gene no longer being produced or only being produced in an inactive form. Within a preferred embodiment, in addition to SEQ ID NO:1 also SEQ ID NO: 5 has been disrupted

Exemplary methods which can be used within the present invention are:
the partial or complete removal from the genome of the gene, the region coding for the protein and/or the promoter or other regions necessary for the expression of the gene (=“deletion”)
the alteration of the DNA sequence of the coding region so that a shortened protein (=generation of a stop codon) and/or a protein with an altered amino acid sequence is produced which can no longer perform the function of the unchanged protein (=“mutation”)
the modification of the DNA sequence of the promoter or other regions necessary for the expression of the gene, so that the gene is no longer transcribed (=no RNA and therefore no protein is produced)
the expression of RNA with a sequence complementary to that of the target gene. This leads to hybridization (=pairing of complementary sequences) of the two RNAs and to a degradation of this double-stranded RNA. As a result, no RNA of the target gene is available for protein synthesis (=RNA interference).

Within the present invention SEQ ID NO:1 and SEQ ID NO: 5 are defined within the sequence protocol.

It is to be understood that any embodiment and preferred embodiment defined within the description applies to a filamentous fungus cell wherein only SEQ ID NO: 1 has been disrupted but also to a filamentous fungus cell wherein in addition to SEQ ID NO: 1 SEQ ID NO: 5 has been disrupted.

Mixing according to step (c) of the inventive process of the present invention is carried out for a time period from 1 minute to 10 days, preferably from 10 hours to 7 days, further preferred from 24 hours to 5 days, preferably under constant stirring with a power input from 150 to 20000 W/m³ and more preferably from 500 to 15000 W/m 3 and under oxygen controlled conditions. The average dissolved oxygen level is preferably selected from 0.01% to 80%, preferred from 0.1% to 50%, particularly preferred from 5% to 30% and most preferred from 12% to 28%. Within a particularly preferred embodiment, the dissolved oxygen level is controlled by a stirrer or compressed air flow or internal reactor pressure or a combination of two or three of these measures. Furthermore, mixing according to step (c) of the inventive process is carried out at a temperature of from 20 to 35° C., preferably at a temperature of from 21 to 34° C. wherein a temperature selected from the range of from 22 to 33° C. is also preferred.

“Mixing” according to step (c) of the process of the present invention is preferably conducted in a batch mode (discontinuous), in a fed-batch mode or in a continuous mode. Most preferably, the inventive process is conducted in a fed-batch mode.

“Obtaining” according to step (d) of the inventive process is preferably carried out by harvesting the technical enzyme composition at the end of the time period applied for mixing during step (c) as it is without further treatment.

Within another preferred embodiment of the present invention, the inventive process further contains the step (e): subjecting the technical enzyme composition according to step d) to a purification method. The purification according to step (e) can be carried out by any measure known to a person skilled in the art as suitable for the inventive purpose. Suitable purification methods are selected from the group consisting of filtration (ultrafiltration, microfiltration, nanofiltration, depth filtration, sterile filtration, filter press), centrifugation, decantation, flotation, chromatographic separation, adsorption, electrodialysis, extraction, precipitation, crystallisation, spray drying, granulation, coating, extrusion or combinations thereof. Preferred are filter-based solid-liquid separations. It is further particularly preferred to use a filter press. The residues after the filtration should have a minimal solid content of 20% (wt./wt.), preferably 25% (wt./wt.), particularly preferred 30% (wt./wt.) and most preferred 40% (wt./wt.) solid content. In case the process according to the present invention involves solid-liquid separation as purification, the technical enzyme composition obtained according to step (d) of the inventive process is considered to be the liquid fraction.

Within a preferred embodiment of the inventive process, the process further comprises step
(ai) sterilization of the fermentation medium according to step (a).

Sterilization can thereby be carried out by any means or measure known to a person skilled in the art as suitable for the inventive purpose. Within a preferred embodiment, sterilization is carried out by filtration, such as but not limited to membrane filtration processes or by ultra high temperature heating. A combination of two or more sterilization methods is also possible, however, it is particularly preferred to only apply ultra high temperature heating (also referred to as UHT). The UHT treatment is preferably carried out at a temperature of from 100 to 155° C. and for a duration of from 10 to 30 seconds, more preferred at a temperature of from 120 to 140° C. for a duration of from 10 to 20 seconds.

Within another aspect, the present invention relates to a filamentous fungus cell wherein SEQ ID NO:1 has been disrupted. The term “wherein SEQ ID NO:1 has been disrupted” relates to any filamentous fungus cell, wherein SEQ ID NO:1 is no longer contained or no longer functioning and/or wherein the genome of the filamentous fungus cell contains a disrupted SEQ ID NO: 1 gene. Disruption of SEQ ID NO:1 can be carried out by any means and measure known to a person skilled in the art to be suitable for the inventive purpose. Possible and preferred methods and measures have been defined within the description. Within a preferred embodiment, SEQ ID NO:1 has been disrupted by deletion, mutation, modification of a promotor or any other regulatory sequence, generation of a stop codon or RNA interference. The term “filamentous fungus cell” has been defined within the description. All definitions given apply.

Within a preferred embodiment, in addition to the disruption of SEQ ID NO: 1 also SEQ ID NO: 5 has been disrupted. Possible and preferred methods and measures have been defined within the description.

It is to be understood that any embodiment and preferred embodiment defined within the description applies to a filamentous fungus cell wherein only SEQ ID NO: 1 has been disrupted but also to a filamentous fungus cell wherein in addition to SEQ ID NO: 1 SEQ ID NO: 5 has been disrupted.

Within a preferred embodiment, the filamentous fungus cell is a genetically modified filamentous fungus cell with the ability to express at least one heterologous hydrolase enzyme. Such genetically modified filamentous fungus cell has been defined within the description. Within a particularly preferred embodiment of the present invention, the filamentous fungus cell is a genetically modified filamentous fungus cell wherein the filamentous fungus cell comprises at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence.

In another aspect the present invention relates to a technical enzyme composition produced according to the process as defined before.

In a further aspect the present invention relates to the use of a filamentous fungus cell as defined before for the production of a technical enzyme composition as defined before.

### GENERALLY PREFERRED EMBODIMENTS

In the following, generally preferred embodiments of the present invention are listed which do not limit the scope of the invention and/or scope of the claims in any respect. The generally preferred embodiments illustrate particularly suitable embodiments for the production of technical enzyme composition by the filamentous fungus Trichoderma reesei.

### Generally Preferred Embodiment 1

Process for production of a technical enzyme composition, comprising the following steps:
(a) providing a fermentation medium with a glucose content of from 5 to 550 g/L or from 5 to 450 g/L;
(b) addition of at least one Trichoderma reesei cell wherein SEQ ID NO:1 has been disrupted;
(c) mixing of the fermentation medium and the at least one filamentous fungus cell for a time period of from 1 minute to 10 days at a temperature of from 20 to 35° C.;
(d) obtaining a technical enzyme composition.

Generally preferred embodiment 2 Process for production of a technical enzyme composition, comprising the following steps:
(a) providing a fermentation medium with a glucose content of from 5 to 550 g/L or from 5 to 450 g/L and wherein the fermentation medium further contains xylose and wherein the glucose to xylose ratio is selected from the range of from 1 to 3.5;
(b) addition of at least one Trichoderma reesei cell wherein SEQ ID NO:1 has been disrupted;
(c) mixing of the fermentation medium and the at least one filamentous fungus cell fora time period of from 1 minute to 10 days at a temperature of from 20 to 35° C.;
(d) obtaining a technical enzyme composition.

### Generally Preferred Embodiment 3

Process for production of a technical enzyme composition, comprising the following steps:
(a) providing a fermentation medium with a glucose content of from 5 to 550 g/L or from 5 to 450 g/L and wherein the fermentation medium is free from cellulose, hemicellulose, gluco-oligosaccharides and/or sophorose;
(b) addition of at least one Trichoderma reesei cell wherein SEQ ID NO:1 has been disrupted;
(c) mixing of the fermentation medium and the at least one filamentous fungus cell for a time period of from 1 minute to 10 days at a temperature of from 20 to 35° C.;
(d) obtaining a technical enzyme composition.

Generally preferred embodiment 4 Process for production of a technical enzyme composition, comprising the following steps:
(a) providing a fermentation medium with a glucose content of from 5 to 550 g/L or from 5 to 450 g/L and a cellulose content of from 0.01 g/L to 1 g/L;
(b) addition of at least one Trichoderma reesei cell wherein SEQ ID NO:1 has been disrupted;
(c) mixing of the fermentation medium and the at least one filamentous fungus cell for a time period of from 1 minute to 10 days at a temperature of from 20 to 35° C.;
(d) obtaining a technical enzyme composition.

Generally preferred embodiment 5 Process for production of a technical enzyme composition, comprising the following steps:
(a) providing a fermentation medium with a glucose content of from 5 to 550 g/L or from 5 to 450 g/L;
(b) addition of at least one Trichoderma reesei cell wherein SEQ ID NO:1 has been disrupted by deletion, mutation, modification of a promotor or any other regulatory sequence, generation of a stop codon or RNA interference and wherein the filamentous fungus cell comprises at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence;
(c) mixing of the fermentation medium and the at least one filamentous fungus cell for a time period of from 1 minute to 10 days at a temperature of from 20 to 35° C.;
(d) obtaining a technical enzyme composition.

Generally preferred embodiment 6 Process for production of a technical enzyme composition, comprising the following steps:
(a) providing a fermentation medium with a glucose content of from 5 to 550 g/L or from 5 to 450 g/L and wherein the fermentation medium is free from sophorose and/or gluco-oligosaccharides;
(b) addition of at least one Trichoderma reesei cell wherein SEQ ID NO:1 has been disrupted by deletion, mutation, modification of a promotor or any other regulatory sequence, generation of a stop codon or RNA interference and wherein the filamentous fungus cell comprises at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 7

Process for production of a technical enzyme composition, comprising the following steps:
(a) providing a fermentation medium with a glucose content of from 5 to 550 g/L or from 5 to 450 g/L wherein the fermentation medium can at least partly originate from chemical, mechanical and/or enzymatic hydrolysis of lignocellulosic biomass;
(b) addition of at least one Trichoderma reesei cell wherein SEQ ID NO:1 has been disrupted;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 8

Trichoderma reesei cell, wherein SEQ ID NO:1 has been disrupted by deletion, mutation, modification of a promotor or any other regulatory sequence, generation of a stop codon or RNA interference, comprising at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence.

### Generally preferred embodiment 9

Trichoderma reesei cell, wherein SEQ ID NO:1 has been disrupted by deletion, mutation, modification of a promotor or any other regulatory sequence, generation of a stop codon or RNA interference, comprising at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase enzyme encoding sequence, at least one heterologous protease enzyme encoding sequence, at least one heterologous isomerase enzyme encoding sequence and/or at least one heterologous lytic polysaccharide monooxygenase enzyme encoding sequence and wherein the at least one heterologous enzyme sequence originates from Acremonium, Aspergillus, Chaetomium, Emericella, Fusarium, Humicola, Hypocrea, Irpex, Magnaporte, Myceliophthora, Neurospora, Penicillium, Rhizopus, Talaromyces, Trichoderma and Trametes.

### Generally Preferred Embodiment 10

Technical enzyme composition produced according to a process as defined by any of generally preferred embodiment 1 to 7.

### Generally Preferred Embodiment 11

Use of a filamentous fungus cell as defined by any of generally preferred embodiments 8 or 9 for the production of a technical enzyme composition.

### Generally Preferred Embodiment 12

Process for production of a technical enzyme composition, comprising the following steps:
(a) providing a fermentation medium with a glucose content of from 5 to 550 g/L or from 5 to 450 g/L;
(b) addition of at least one Trichoderma reesei cell wherein SEQ ID NO:1 and SEQ ID NO: 5 have been disrupted;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 13

Process for production of a technical enzyme composition, comprising the following steps:
(a) providing a fermentation medium with a glucose content of from 5 to 550 g/L or from 5 to 450 g/L and wherein the fermentation medium further contains xylose and wherein the glucose to xylose ratio is selected from the range of from 1 to 3.5;
(b) addition of at least one Trichoderma reesei cell wherein SEQ ID NO:1 and SEQ ID NO: 5 have been disrupted;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 14

Process for production of a technical enzyme composition, comprising the following steps:
(a) providing a fermentation medium with a glucose content of from 5 to 550 g/L or from 5 to 450 g/L and wherein the fermentation medium is free from cellulose, hemicellulose, gluco-oligosaccharides and/or sophorose;
(b) addition of at least one Trichoderma reesei cell wherein SEQ ID NO:1 and SEQ ID NO: 5 have been disrupted;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 15

Process for production of a technical enzyme composition, comprising the following steps:
(a) providing a fermentation medium with a glucose content of from 5 to 550 g/L or from 5 to 450 g/L and a cellulose content of from 0.01 g/L to 1 g/L;
(b) addition of at least one Trichoderma reesei cell wherein SEQ ID NO:1 and SEQ ID NO: 5 have been disrupted;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 16

Process for production of a technical enzyme composition, comprising the following steps:
(a) providing a fermentation medium with a glucose content of from 5 to 550 g/L or from 5 to 450 g/L;
(b) addition of at least one Trichoderma reesei cell wherein SEQ ID NO:1 and SEQ ID NO: 5 have been disrupted by deletion, mutation, modification of a promotor or any other regulatory sequence, generation of a stop codon or RNA interference and wherein the filamentous fungus cell comprises at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 17

Process for production of a technical enzyme composition, comprising the following steps:
(a) providing a fermentation medium with a glucose content of from 5 to 550 g/L or from 5 to 450 g/L and wherein the fermentation medium is free from sophorose and/or gluco-oligosaccharides;
(b) addition of at least one Trichoderma reesei cell wherein SEQ ID NO:1 and SEQ ID NO: 5 have been disrupted by deletion, mutation, modification of a promotor or any other regulatory sequence, generation of a stop codon or RNA interference and wherein the filamentous fungus cell comprises at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 18

Process for production of a technical enzyme composition, comprising the following steps:
(a) providing a fermentation medium with a glucose content of from 5 to 550 g/L or from 5 to 450 g/L wherein the fermentation medium can at least partly originate from chemical, mechanical and/or enzymatic hydrolysis of lignocellulosic biomass;
(b) addition of at least one Trichoderma reesei cell wherein SEQ ID NO:1 and SEQ ID NO: 5 have been disrupted;
(c) mixing of the fermentation medium and the at least one filamentous fungus cell for a time period of from 1 minute to 10 days at a temperature of from 20 to 35° C.;
(d) obtaining a technical enzyme composition.

### Generally Preferred Embodiment 19

Trichoderma reesei cell, wherein SEQ ID NO:1 and SEQ ID NO: 5 have been disrupted by deletion, mutation, modification of a promotor or any other regulatory sequence, generation of a stop codon or RNA interference, comprising at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase encoding sequence, at least one heterologous protease enzyme encoding sequence, at least one heterologous isomerase enzyme encoding sequence and/or at least one heterologous lytic polysaccharide monooxygenase enzyme encoding sequence.

### Generally Preferred Embodiment 20

Trichoderma reesei cell, wherein SEQ ID NO:1 and SEQ ID NO: 5 have been disrupted by deletion, mutation, modification of a promotor or any other regulatory sequence, generation of a stop codon or RNA interference, comprising at least one heterologous beta-glucosidase enzyme encoding sequence, at least one heterologous cellulase enzyme encoding sequence, at least one heterologous xylanase enzyme encoding sequence, at least one heterologous beta-xylosidase enzyme encoding sequence, at least one heterologous pectinase enzyme encoding sequence, at least one heterologous oxidase enzyme encoding sequence, at least one heterologous protease enzyme encoding sequence, at least one heterologous isomerase enzyme encoding sequence and/or at least one heterologous lytic polysaccharide monooxygenase enzyme encoding sequence and wherein the at least one heterologous enzyme sequence originates from Acremonium, Aspergillus, Chaetomium, Emericella, Fusarium, Humicola, Hypocrea, Irpex, Magnaporte, Myceliophthora, Neurospora, Penicillium, Rhizopus, Talaromyces, Trichoderma and Trametes.

### Generally Preferred Embodiment 21

Technical enzyme composition produced according to a process as defined by any of generally preferred embodiment 10 to 18.

### Generally Preferred Embodiment 22

Use of a filamentous fungus cell as defined by any of generally preferred embodiments 19 or 20 for the production of a technical enzyme composition.

### FIGURES AND EXAMPLES

The present invention is described by the following figures and examples. It is thereby emphasized that the figures and examples do not limit the scope of the invention and claims but merely constitute further illustration of the invention, inventive purpose and benefits achieved by the inventive method.

### LIST OF FIGURES

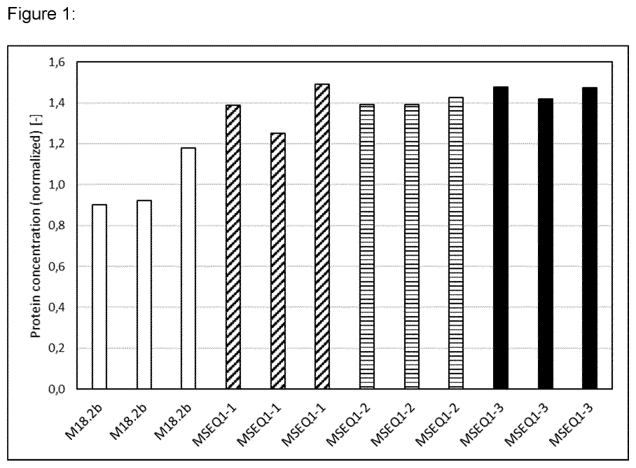

FIG. 1: Protein concentrations in the culture supernatants of pSEQ1M-HygR transformants MSEQ1-1 to -3 and reference strain M18.2b. Values are given in relation to the average protein concentration in the supernatants of the host strain M18.2b which is set to 1.

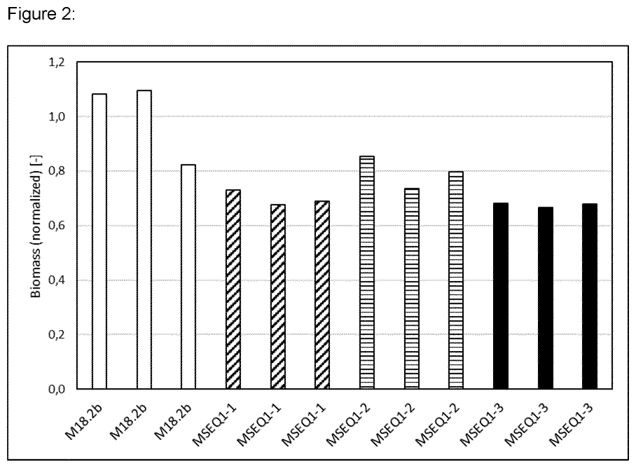

FIG. 2: Biomass concentrations in the culture broths of pSEQ1M-HygR transformants MSEQ1-1 to -3 and reference strain M18.2b. Values are given in relation to the average biomass concentration in the culture broth of the host strain M18.2b which is set to 1.

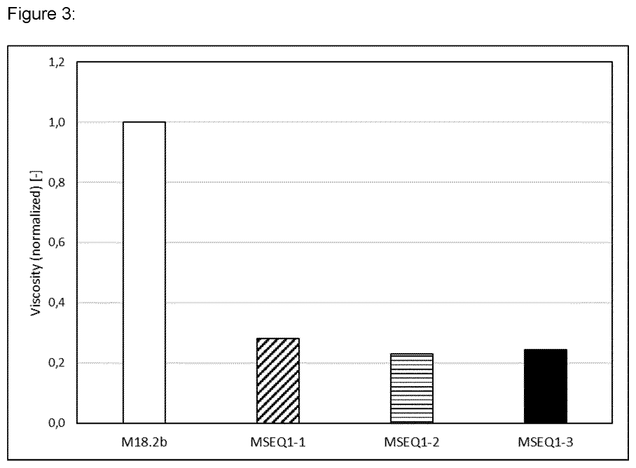

FIG. 3:

Viscosity of culture broths of pSEQ1M-HygR transformants MSEQ1-1 to -3 and reference strain M18.2b. Values are given in relation to the viscosity of the culture broth of the host strain M18.2b which is set to 1.

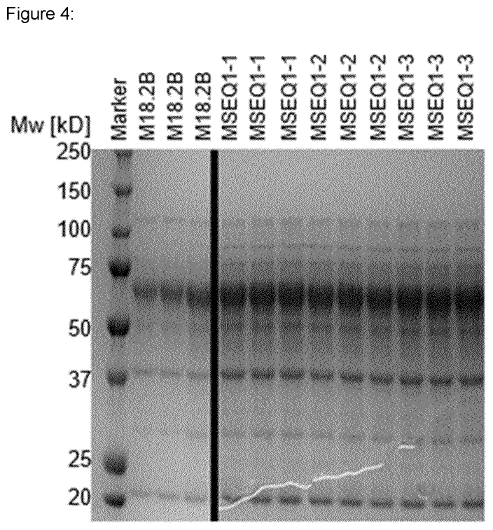

FIG. 4: SDS-PAGE gel of culture supernatants of pSEQ1M-HygR transformants MSEQ1-1 to -3 and reference strain M18.2b.

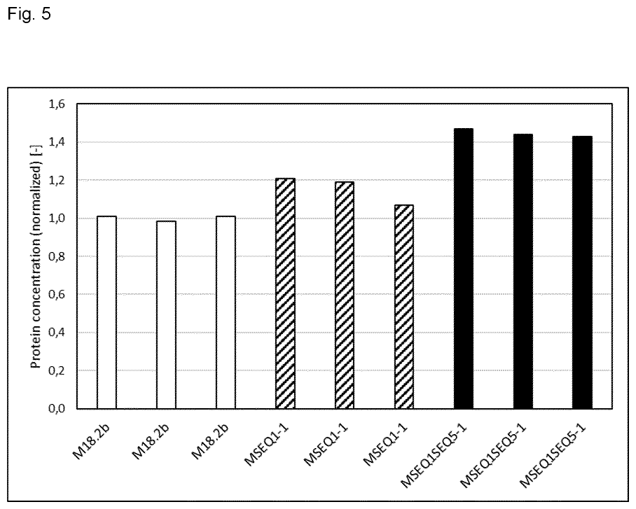

FIG. 5: Protein concentrations in the culture supernatants of MSEQ1-1 based pSEQ5M-amdS transformant MSEQ1SEQ5-1 and reference strains MSEQ1-1 and M18.2b. Values are given in relation to the average protein concentration in the supernatants of the host strain M18.2b which is set to 1.

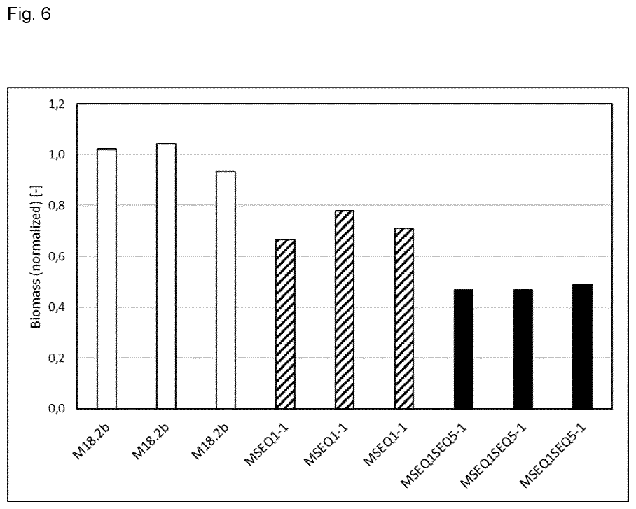

FIG. 6: Biomass concentrations in the culture broths of MSEQ1-1 based pSEQ5M-amdS transformant MSEQ1SEQ5-1 and reference strains MSEQ1-1 and M18.2b. Values are given in relation to the average biomass concentration in the culture broth of the host strain M18.2b which is set to 1.

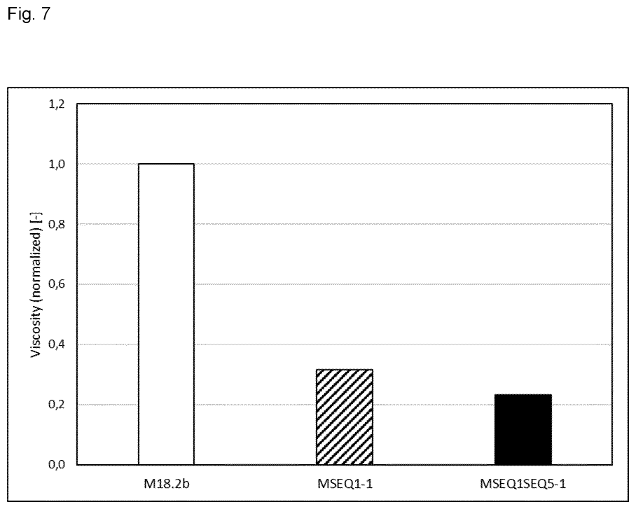

FIG. 7: Viscosity of culture broths of MSEQ1-1 based pSEQ5M-amdS transformant MSEQ1SEQ5-1 and reference strains MSEQ1-1 and M18.2b. Values are given in relation to the viscosity of the culture broth of the host strain M18.2b which is set to 1.

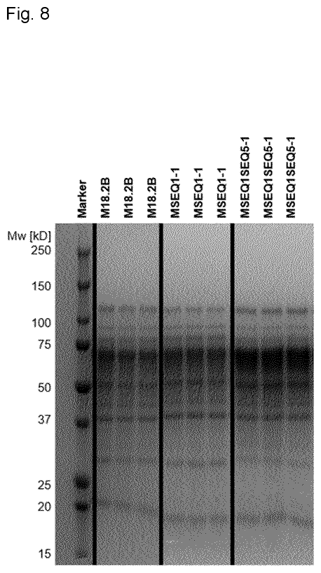

FIG. 8 SDS-PAGE gel of culture supernatants of MSEQ1-1 based pSEQ5M-amdS transformant MSEQ1SEQ5-1 and reference strains MSEQ1-1 and M18.2b.

### GENERAL

The examples describe a way to disrupt the Trichoderma reesei SEQ1 gene by deleting two nucleotides resulting in a frame shift and a change of an amino acid coding codon to a stop codon. They also show the effect of the SEQ1 gene disruption on the protein production, biomass formation and culture broth viscosity of T. reesei and the effect of the disruption of both the SEQ1 and SEQ5 genes on the protein production, biomass formation and culture broth viscosity of T. reesei.

### Example 1: Construction of a SEQ1 Mutation Vector

Standard methods known to those skilled in the art and described e.g. by Sambrook and Russel (Molecular Cloning—A laboratory manual; Cold Spring Harbor Laboratory Press, New York) or by Jansohn et al. (Gentechnische Methoden, Elsevier, München) were used for DNA agarose gel electrophorese, purification of DNA, transformation of Escherichia coli, plasmid propagation and purification, amplification of pieces of DNA by polymerase chain reaction (PCR) and isolation of genomic DNA from Trichoderma reesei. Ligation-independent cloning (LIC) was done essentially as described by Aslanidis and de Jong (1990, Nucleic Acid Res. 18 (20), 6069).

A SEQ1 mutation vector was constructed by fusing the Hygromycin B resistance marker to the SEQ1 3′ flanking region and cloning the fusion product in a plasmid containing a part of the SEQ1 coding region that introduces a mutation encompassing the deletion of the nucleotides G4060 and T4061 (positions according to SEQ ID NO: 1) into the SEQ1 gene.

The Hygromycin B resistance marker cassette (SEQ ID NO:2) had been synthesized by Thermo Scientific. Primers hygrfw (5′-TGCAAGGCGATTAAGTTGGG-3′; SEQ ID NO: 6) and hygrrv (5′-CGGCGAGGATCTTTCCTCGCTGCTTCTCTCAACAGACAAGAGCCCTATAACTTC-3′; SEQ ID NO: 7) were used to amplify the approximately 2.6 kb long cassette (annealing temperature: 63.2° C., elongation time: 1 min, 30 cycles) using phusion polymerase from Thermo Scientific.

Genomic DNA from Trichoderma reesei M18.2b (DSM 19984) was isolated and used as a template together with the primers SEQ1fl3fw (5′-TTGTCAACGCCATCTTGAGC-3′; SEQ ID NO: 8) and SEQ1fl3ry (5′-ACCAACCAGTCCATCCTCTG-3′; SEQ ID NO: 9) to amplify an approximately 2.2 kb 3′ flanking fragment of SEQ1 (annealing temperature: 64.5° C., elongation time: 1 min 15 sec, 30 cycles) using phusion polymerase from Thermo Scientific.

The PCR-amplified hygromycin B resistance marker cassette and SEQ1 3′ flanking region were purified and fused using phusion polymerase from Thermo Scientific and the primers fust (5′-AAACCAGACAGACAGTATACGACTCACTATAGGGCG -3′; SEQ ID NO: 10), fus2 (5′-GTTAACAGACAAGAGCCCGAAGTTATTCGGGTAGTAGAGTTTGAAAGGGG -3′; SEQ ID NO: 11) and fus3 (5′-AGAGAGGAGAGACAGTGTTAACAGACAAGAGCCCGAAG -3′; SEQ ID NO: 12).

Approximately 100 ng of both templates, 20 μM of primers fust and fus3 and 2 μM of primer fus2 were used. The PCR consisted of 10 initial cycles of 10 sec at 98° C., 30 sec at 65° C. and 1 min 20 sec at 72° C. followed by cooling to 10° C. Then the primers were added, followed by a 30 sec hold at 98° C. and 30 cycles of 10 sec at 98° C., 30 sec at 61.5° C. and initial 2 min 5 sec at 72° C. with the 72° C. incubation being extended by 5 sec per cycle. The PCR was concluded by a 10 sec hold at 72° C. and cooling to 10° C.

The approx. 4.1 kb long fusion PCR product was purified and cloned into a PshAI-linearized pUC19-derived plasmid (SEQ ID NO: 3) that contained a LIC reception site instead of the multiple cloning site. The linearized vector was treated with T4 DNA polymerase in the presence of dTTP. The fusion PCR product was treated with T4 DNA polymerase in the presence of dATP. T4 DNA polymerase treated vector and fusion PCR amplicon were mixed and annealed as described by Aslanidis and de Jong. The LIC assay was then transformed in chemically competent Escherichia coli XL1-Blue cells (Agilent), plated on LB-Agar plates containing 100 mg·l⁻¹ ampicillin (LB-Amp) and incubated at 37° C. for 24 h. Colonies were picked from the agar plates using toothpicks, transferred into liquid LB-Amp medium and incubated at 37° C. for 24 h with shaking (250 RPM). Plasmid DNA was isolated and integration of the insert was verified by digestion with HpaI. Plasmid clones were verified by Sanger sequencing and one plasmid with correct sequence was designated pSEQ1-3fl-HygR.

Plasmid pSEQ1flank5 (synthesized at Thermo Scientific; SEQ ID NO: 4), containing a modified part of the SEQ1 gene that introduces a mutation encompassing the deletion of the nucleotides G4060 and T4061 (positions according to SEQ ID NO: 1) into the SEQ1 gene was digested with Srfl (New England Biolabs).

The Hygromycin resistance marker—SEQ1 3′ flanking region fragment (approx. 4.0 kb) was released from pSEQ1-3fl-HygR by restriction digestion with HpaI. The Sill-linearized vector pSEQ1flank5 was treated with T4 DNA polymerase in the presence of dTTP. The 4.0 kb HpaI fragment from pSEQ1-3fl-HygR was treated with T4 DNA polymerase in the presence of dATP. T4 DNA polymerase treated vector and insert were mixed and annealed as described in by Aslanidis and de Jong. The assay was then transformed in chemically competent Escherichia coli XL1-Blue cells (Agilent), plated on LB-Agar plates containing 100 mg·l⁻¹ ampicillin (LB-Amp) and incubated at 37° C. for 24 h. Colonies were picked from the agar plates using toothpicks, transferred into liquid LB-Amp medium and incubated at 37° C. for 24 h with shaking (250 RPM). Plasmid DNA was isolated and integration of the insert was verified by digestion with XmnI. Plasm id clones were verified by Sanger sequencing and one plasmid with correct sequence was designated pSEQ1M-HygR.

### Example 2: Transformation of the SEQ1 Mutation Vector into Trichoderma reesei

Vector pSEQ1M-HygR was digested with XmnI (New England Biolabs) according to the manufacturer's instructions and the mutation cassette (6.0 kb) was purified by agarose gel electrophoresis and with the Wizard PCR purification kit from Promega. Trichoderma reesei M18.2b (DSM 19984) was transformed with the digested vector essentially as described in Penttilä et al (1987) Gene 61: 155-164 or Gruber et al (1990) Curr Genet 18: 71-76. The transformants were selected on potato dextrose agar plates containing 100 mg·l⁻¹ of Hygromycin B and 1 M sorbitol and purified by singularisation. Conidia stocks of the purified strains were prepared by growing them on potato dextrose agar plates at 30° C. until the plates were covered with spores. The conidia were harvested with sterile sodium chloride (0.9 g·l⁻¹)-Triton X-100 (0.01 g·l⁻¹) solution, adjusted to OD₆₀₀=10 with sterile water, supplemented with glycerol to a final concentration of 50 g·l⁻¹ and stored at −80° C. In order to determine the conidia titer, aliquots of the stocks were thawed, appropriately diluted in potato dextrose broth and plated on potato dextrose agar containing 1 g·l⁻¹ of Triton X-100. The plates were incubated at 30° C. for 4 days and then the colonies on the plates were counted.

Genomic DNA was isolated from the mycelium of the transformants and the host strain. The integration of the SEQ1 mutation cassette at the intended locus was verified by PCR using phusion polymerase from Thermo Fisher Scientific according to the manufacturer's instructions, genomic DNA from the transformants as template and primers SEQ1MKOfw (5′-ATGTGCTAGGATTGTACGAG -3′; SEQ ID NO: 13) and SEQ1MKO1rv (5′-ATAATAGCTCATGGTCTCAC -3′; SEQ ID NO: 14) (annealing temperature: 57.3° C., elongation time: 1 min 20 sec, 30 cycles) or primers SEQ1MKOfw (5′-ATGTGCTAGGATTGTACGAG -3′; SEQ ID NO: 13) and SEQ1MKO2ry (5′-TTGACAAAGGCCACAATATC -3′; SEQ ID NO: 15) (annealing temperature: 59.3° C., elongation time: 1 min 15 sec, 30 cycles), respectively. A 2.6 kb band with primers SEQ1MKOfw and SEQ1MKO1rv indicates the integration of the mutation cassette at the SEQ1 locus, while a 2.4 kb band with primers SEQ1MKOfw and SEQ1MKO2ry indicates that the SEQ1 locus is still native (i.e. this band was not expected with genomic DNA from transformants that had integrated the pSEQ1M-HygR fragment at the intented locus). Genomic DNA from strain M18.2b was also tested as a control. In order to verify that the intended mutation had been inserted into the SEQ1 ORF, the amplicon obtained with primers SEQ1MKOfw and SEQ1MKO1rv was sequenced using primer M1Seq-01 (5′-ATCGCTACTTCTTTGTTCAG -3′; SEQ ID NO: 16) and M1Seq-02 (5′-CAGCTTGGAATACAGCACTG -3′; SEQ ID NO: 17).

Three transformants containing the mutation from pSEQ1M-HygR in the SEQ1 ORF were named MSEQ1-1 to -3.

### Example 3: Growth of the SEQ1 Mutation Strains in Shake Flasks

The strains MSEQ1-1 to -3 and M18.2b were grown in shake flasks in Hydrolysate Medium 1. Hydrolysate Medium 1 contains (g·l⁻¹):

| | Concentration |
|---|---|
| Name | [g/l] |
| Acetic acid | 0.34 |
| Calcium | 0.12 |
| Chloride, water | 0.15 |
| soluble | |
| Copper | 0.0001 |
| Fat (HCl soluble) | 0.001 |
| Furfural | 0.003 |
| Glucose | 6.5 |
| Glycerol | 0.009 |
| HMF | 0.006 |
| Iron | 0.004 |
| Magnesium | 0.048 |
| Manganese | 0.002 |
| Na-D/L-Lactat | 0.097 |
| Nitrogen, soluble | 0.85 |
| Phosphorus | 0.48 |
| Phthalate | 8.2 |
| Potassium | 3.2 |
| Sodium | 0.015 |
| Sulfur | 0.86 |
| Xylose | 3.6 |
| Zinc | 0.001 |

The medium was adjusted to pH 5.5 with HCl or NaOH and sterilized by autoclaving (20 min at 121° C.).

15 ml of the medium were distributed into 50 ml Erlenmeyer shake flasks under a sterile hood. Conidia stocks of strains MSEQ1-1 to -3 and M18.2b were thawed, conidia suspensions corresponding to 2.5*10⁵ conida were pipetted into the Erlenmeyer flasks with the medium under a sterile hood and the flasks were closed with rubber foam caps. Three flasks were inoculated per strain. The flasks were incubated at 30° C. with shaking (250 RPM) for 6 days. After 6 days, the cultures were poured into 15 ml tubes. Aliquots were removed, centrifuged (3220×g, 4° C., 15 min) and the supernatants stored at 4° C., while the remaining culture broth was used for determination of the biomass and viscosity (see below).

### Example 4: Characterization of the Culture Supernatants and Broths: Protein Concentration, SDS-PAGE, Biomass, Viscosity

Protein concentrations in the centrifuged culture supernatants of strains MSEQ1-1 to -3 and M18.2b were measured using the Quick Start™ Bradford reagent (BioRad) and BSA standard solutions (BioRad) according to the supplier's instructions. The results of the measurements are shown in FIG. 1. Values are given in relation to the average protein concentration in the supernatants of the host strain M18.2b which is set to 1. It is obvious from these data that strains MSEQ1-1 to -3 produce significantly more protein than the host strain M18.2b.

For biomass determination, Whatman™ filter discs (P1) were dried at 60° C. until their weight remained constant for 24 h, cooled to room temperature and weighed. Culture broths of strains MSEQ1-1 to -3 and M18.2b were filtered using those dried filter discs and the mycelia were washed with at least ten times the broth's volume of deionized water. Then the filter discs with the mycelia were dried at 60° C. until their weight remained constant for 24 h. The filter discs with the dried mycelia were weighed. The biomass concentration in the culture broth was then calculated by subtracting the mass of the dried filter disc from the mass of the dried filter disc with the mycelia and then dividing that value by the volume of the culture broth that had been filtered. The results of the measurements are shown in FIG. 2. Values are given in relation to the average biomass concentration in the culture broth of the host strain M18.2b which is set to 1. It is obvious from these data that strains MSEQ1-1 to -3 produce significantly less biomass than the host strain M18.2b.

The viscosity of the culture broths of strains MSEQ1-1 to -3 and M18.2b was measured using a Malvern Kinexus Lab+ KNX2110 rotational rheometer with the Vane tool (4Vnn:CUPnn) according to the manufacturer's instructions. The measurements were taken at a temperature of 20° C. and at a rotation velocity of 18.11 RPM (“rotations per minute”). The viscosities are depicted in FIG. 3 and are presented in relation to the viscosity of the culture broth of strain M18.2b, which is set to 1. It is obvious from these data that the viscosity of the culture broths produced with MSEQ1-1 to -3 is significantly lower than that of the host strain M18.2b.

SDS-PAGE analysis of the centrifuged culture supernatants of strains MSEQ1-1 to -3 and M18.2b was done using methods known to those skilled in the art (e.g. described by Jansohn et al. (Gentechnische Methoden, Elsevier, München)) and the Criterion XT system (BioRad). Equal volumes of culture supernatants were loaded in each lane. Precision Plus Protein™ All Blue Standards (BioRad) was used as protein size reference. The gel image is shown in FIG. 4. A person skilled in the art will recognize that the protein pattern of MSEQ1-1 to -3 is indistinguishable from that of the host strain M18.2b.

### Example 5: Construction of a SEQ5 Mutation Vector

Standard methods known to those skilled in the art and described e.g. by Sambrook and Russel (Molecular Cloning—A laboratory manual; Cold Spring Harbor Laboratory Press, New York) or by Jansohn et al. (Gentechnische Methoden, Elsevier, MOnchen) were used for DNA agarose gel electrophorese, purification of DNA, transformation of Escherichia coli, plasmid propagation and purification, amplification of pieces of DNA by polymerase chain reaction (PCR) and isolation of genomic DNA from Trichoderma reesei and Emericella nidulans. Ligation-independent cloning (LIC) was done essentially as described by Aslanidis and de Jong (1990, Nucleic Acid Res. 18 (20), 6069).

A SEQ5 mutation vector was constructed by fusing the Emericella nidulans amdS gene to the SEQ5 5′ and 3′ flanking regions and cloning the fusion product in a pUC19-derived plasmid.

The SEQ5 5′ flanking region was amplified by PCR using genomic DNA from Trichoderma reesei M18.2b (DSM 19984) as template, primers SEQ5M5fw (5C-GACTCTCTATCTGCATCAAC-3; SEQ ID NO: 18) and SEQ5M5ry (5c-TGACCTGGAAAGCTTTCAATGTAGAGGTAGACTAGTCAAAGAAGACATCACGAC-3′; SEQ ID NO: 19) and phusion polymerase from Thermo Fisher Scientific according to the manufacturer's instructions (annealing temperature: 64.8° C., elongation time: 1 min 25 sec, 30 cycles). The amplicon (2.7 kb) was purified using the Wizard PCR purification kit from Promega.

The SEQ5 3′ flanking region was amplified by PCR using genomic DNA from Trichoderma reesei M18.2b (DSM 19984) as template, primers SEQ5M3fw (5c-CGCATGGTGGGCGTCGTGATGTCTTCTTTGACTAGTCTACCTCTACATTGAAAG C -3; SEQ ID NO: 20) and SEQ5M3ry (5′-GATTACCTGTCAAGTCTATG -3; SEQ ID NO: 21) and phusion polymerase from Thermo Fisher Scientific according to the manufacturer's instructions (annealing temperature: 62.4° C., elongation time: 1 min sec, 30 cycles). The amplicon (2.7 kb) was purified using the Wizard PCR purification kit from Promega.

The SEQ5 5′ and 3′ flanking regions were fused by PCR using Phusion polymerase (Thermo Fisher Scientific) and the buffer and dNTP solution provided with the polymerase. 100 ng purified SEQ5 5′ PCR amplicon, 100 ng purified SEQ5 3′ amplicon, 10 μl 5× Phusion HF buffer, 1 μl 10 mM dNTP solution, 1 U Phusion polymerase and PCR grade water up to a final volume of 48 μl were mixed. The mixture was first incubated at 98° C. for 30° C. and then subjected to 10 cycles of 10 sec at 98° C., 30 sec at 65° C. and 2 min 40 sec at 72° C. and then cooled to 10° C. Then 1 μl of a 20 μM solution of primer SEQ5Mnestfw (5′-GACAGTCCTGCAGGAGTCACTGCCTTTGAAAG -3′; SEQ ID NO: 22) and 1 μl of a μM solution of primer SEQ5Mnestry (5′-GACAGTCCTGCAGGTGTAAGGATAAAGGACGAC -3′; SEQ ID NO: 23) were added and the mixture was incubated at 98° C. for 30 sec and then subjected to 30 cycles of 10 sec at 98° C., 30 sec at 66.2° C. and 1 min 20 sec at 72° C. The incubation time at 72° C. was increased by 5 sec per cycle. Finally the mixture was incubated at 72° C. for 10 min and then cooled to 10° C. The amplicon (5.2 kb) was purified using the Wizard PCR purification kit from Promega.

The purified SEQ5 5′-3′ flank fusion product was digested with SbfI (New England Biolabs) according to the manufacturer's instructions and purified using the Wizard PCR purification kit from Promega.

Plasmid pUC19 (New England Biolabs) was digested with SbfI (New England Biolabs) according to the manufacturer's instructions and purified using the Wizard PCR purification kit from Promega.

The SbfI-digested SEQ5 5′-3′ flank fusion product and pUC19 were ligated using the “Mighty Mix” DNA ligation kit (Takara) according to the manufacturer's instructions using a molar insert/vector ratio of 5 to 1. The ligation mixture was transformed into Escherichia coli Mach 1 (Thermo Fisher Scientific) and plated on LB agar plates containing 100 mg·l⁻¹ ampicillin. After 20 h of incubation at 37° C. colonies were picked from the plate and used to inoculate 3 ml of LB liquid medium with 100 mg·l⁻¹ ampicillin. After 20 h of incubation at 37° C. plasmid DNA was isolated and digested with SbfI to identify clones containing the insert. A plasmid containing the insert was designated pSEQ5-5′-3′.

Plasm id pSEQ5-5′-3′ was digested with SpeI (New England Biolabs) according to the manufacturer's instructions and purified using the Wizard PCR purification kit from Promega. 1 μl each of 10 μM solutions of oligonucleotides LIC1fw (5′-CTAGGAGTTCTGCCTTGGGTTTAAACGAGAGAAAGACTC -3′; SEQ ID NO: 24) and LIC1rv (5′-CTAGGAGTCTTTCTCTCGTTTAAACCCAAGGCAGAACTC-3′; SEQ ID NO: 25) were mixed, put in a PCR cycler and cooled from 70 to 20° C. over the course of 2 h. Then the oligonucleotide mixture was mixed with 750 ng of purified, SpeI-digested pSEQ5-5′-3′, 1 μl 10×T4 Ligase buffer (Promega), 1 μl 500 g/l PEG3350, 1 μl T4 DNA Ligase (5 U/μl; Thermo Fisher Scientific) and 2 μl of PCR-grade water. The mixture was incubated for 1 h at 20° C., purified using the Wizard PCR purification kit from Promega and the DNA eluted in 50 μl of PCR-grade water. This solution was supplemented with 6 μl of Taq Polymerase buffer (Promega) and PCR-grade water was added to a final volume of 60 μl. The mixture was then transformed into Escherichia coli Mach 1 (Thermo Fisher Scientific) and plated on LB agar plates containing 100 mg·l⁻¹ ampicillin. After 20 h of incubation at 37° C. colonies were picked from the plate and used to inoculate 3 ml of LB liquid medium with 100 mg·l⁻¹ ampicillin. After 20 h of incubation at 37° C. plasmid DNA was isolated and digested with PmeI and SspI (New England Biolabs) according to the manufacturer's instructions to identify clones containing the insert. A plasm id containing the insert was designated pSEQ5-5′-3′-LIC.

Plasmid pSEQ5-5′-3′-LIC was digested with PmeI (New England Biolabs) according to the manufacturer's instructions and purified using the Wizard PCR purification kit from Promega.

The E. nidulans amdS gene including the promotor and the terminator (SEQ ID NO: 26) was amplified by PCR using genomic DNA from E. nidulans strain CBS 124.59 as template, primers SEQ5MamdSfw (5′-GTTCTGCCTTGGGTTTAGGATGTACGACGTATATCC -3′; SEQ ID NO: 27) and SEQ5MamdSry (5′-GTCTTTCTCTCGTTTATGATGTCTATTGGAAGAAAACTTGG-3′; SEQ ID NO: 28) and phusion polymerase from Thermo Fisher Scientific according to the manufacturer's instructions (annealing temperature: 56.9° C., elongation time: 1 min 45 sec, 30 cycles). The amplicon (3.4 kb) was purified using the Wizard PCR purification kit from Promega.

The PCR-amplified amdS gene was fused with PmeI-digested pSEQ5-5′-3′-LIC using ligation independent cloning (LIC). The linearized vector was treated with T4 DNA polymerase in the presence of dATP. PCR-amplified amdS was treated with T4 DNA polymerase in the presence of dTTP. T4 DNA polymerase treated vector and amdS were mixed and annealed as described by Aslanidis and de Jong (1990, Nucleic Acid Res. 18 (20), 6069). The assays were then transformed in chemically competent Escherichia coli Mach 1 (Thermo Fisher Scientific), plated on LB-Agar plates containing 100 mg·l⁻¹ ampicillin and incubated at 37° C. for 24 h. Colonies were picked from the agar plates using toothpicks, transferred into liquid LB medium containing 100 mg·l⁻¹ ampicillin and incubated at 37° C. for 24 h with shaking (250 RPM). Plasmid DNA was isolated and integration of the insert was verified by digestion with SbfI. Plasmid clones were verified by Sanger sequencing and one plasmid with correct sequence was designated pSEQ5M-amdS.

### Example 6: Transformation of the SEQ5 Mutation Vector into Trichoderma reesei

Vector pSEQ5M-amdS was digested with SbfI (New England Biolabs) according to the manufacturer's instructions and the mutation cassette (8.6 kb) was purified by agarose gel electrophoresis and with the Wizard PCR purification kit from Promega. Trichoderma reesei MSEQ1-1 was transformed with the digested vector essentially as described in Penttilä et al (1987) Gene 61: 155-164 or Gruber et al (1990) Curr Genet 18: 71-76. The transformants were selected on acetamide selection plates (containing in g·l⁻¹: Acetamide 0.6, CaCl₂*2H₂O 0.3, Agar Noble 15, CsCl 2.5, FeSO₄*7H₂O 0.005, CuSO₄*5H₂O 0.0001, Glucose 20, KH₂PO₄ 15, MgSO₄*7H₂O 0.3, MnSO₄*H₂O 0.0016, Sorbitol 182, ZnSO₄*7H₂O 0.0014; adjusted to pH 5.5) and purified by singularisation. Conidia stocks of the purified strains were prepared by growing them on potato dextrose agar plates at 30° C. until the plates were covered with spores. The conidia were harvested with sterile sodium chloride (0.9 g·l⁻¹)-Triton X-100 (0.01 g·l⁻¹) solution, adjusted to OD₆00=10, supplemented with 50 g·l⁻¹ of glycerol and stored at −80° C. In order to determine the conidia titer, aliquots of the stocks were thawed, appropriately diluted in potato dextrose broth and plated on potato dextrose agar containing 1 g·l⁻¹ of Triton X-100. The plates were incubated at 30° C. for 4 days and then the colonies on the plates were counted.

Genomic DNA was isolated from the mycelium of the transformants and the host strain. The integration of the SEQ5 mutation cassette at the intended locus was verified by PCR using phusion polymerase from Thermo Fisher Scientific according to the manufacturer's instructions, genomic DNA from the transformants as template and primers SEQ5MKO1fw (5′-ACTCTCTATCTGCATCAAC -3′; SEQ ID NO: 29) and SEQ5MKO1rv (5′-GATCCCCGATTTCTTTGG -3′; SEQ ID NO: 30) (annealing temperature: 56.9° C., elongation time: 1 min 20 sec, 30 cycles) and primers SEQ5MKO2fw (5′-TGATGTGCTTGATATTGGGC -3′; SEQ ID NO: 31) and SEQ5MKO2ry (5′-CTCCATCGCTCAACTATGTG -3′; SEQ ID NO: 32) (annealing temperature: 57.5° C., elongation time: 1 min 15 sec, 30 cycles). A 3.9 kb band with primers SEQ5MKO1fw and SEQ5MKO1rv indicates the integration of the mutation cassette at the SEQ5 locus thereby replacing the SEQ5 coding region, while SEQ5MKO2fw and SEQ5MKO2ry (1.2 kb amplicon) amplify a part of the SEQ5 gene replaced by pSEQ5M-amdS and therefore only give a band when the SEQ5 gene is still present. Genomic DNA from strain MSEQ1-1 was also tested as a control.

A MSEQ1-1-derived strain that had integrated the mutation cassette from pSEQ5M-amdS at the SEQ5 locus and thereby replaced the SEQ5 gene was named MSEQ1SEQ5-1.

### Example 7: Growth of the SEQ1SEQ5 Mutation Strain in Shake Flasks

The strains MSEQ1SEQ5-1, MSEQ1-1 and M18.2b were grown in shake flasks in Hydrolysate Medium 1. Hydrolysate Medium 1 contains (g·l⁻¹):

| | Concentration |
|---|---|
| Name | [g/l] |
| Acetic acid | 0.34 |
| Calcium | 0.12 |
| Chloride, water | 0.15 |
| soluble | |
| Copper | 0.0001 |
| Fat (HCl soluble) | 0.001 |
| Furfural | 0.003 |
| Glucose | 6.5 |
| Glycerol | 0.009 |
| HMF | 0.006 |
| Iron | 0.004 |
| Magnesium | 0.048 |
| Manganese | 0.002 |
| Na-D/L-Lactat | 0.097 |
| Nitrogen, soluble | 0.85 |
| Phosphorus | 0.48 |
| Phthalate | 8.2 |
| Potassium | 3.2 |
| Sodium | 0.015 |
| Sulfur | 0.86 |
| Xylose | 3.6 |
| Zinc | 0.001 |

The medium was adjusted to pH 5.5 with HCl or NaOH and sterilized by autoclaving (20 min at 121° C.).

15 ml of the medium were distributed into 50 ml Erlenmeyer shake flasks under a sterile hood. Conidia stocks of strains MSEQ1SEQ5-1, MSEQ1-1 and M18.2b were thawed, conidia suspensions corresponding to 2.5*10⁵ conida were pipetted into the Erlenmeyer flasks with the medium under a sterile hood and the flasks were closed with rubber foam caps. Three flasks were inoculated per strain. The flasks were incubated at 30° C. with shaking (250 RPM) for 6 days. After 6 days, the cultures were poured into 15 ml tubes. Aliquots were removed, centrifuged (3220×g, 4° C., 15 min) and the supernatants stored at 4° C., while the remaining culture broth was used for determination of the biomass and viscosity (see below).

### Example 8: Characterization of the Culture Supernatants and Broths: Protein Concentration, SDS-PAGE, Biomass, Viscosity

Protein concentrations in the centrifuged culture supernatants of strains MSEQ1SEQ5-1, MSEQ1-1 and M18.2b were measured using the Quick Start™ Bradford reagent (BioRad) and BSA standard solutions (BioRad) according to the supplier's instructions. The results of the measurements are shown in FIG. 5. Values are given in relation to the average protein concentration in the supernatants of strain M18.2b which is set to 1. It is obvious from these data that strain MSEQ1SEQ5-1 produces significantly more protein than strains MSEQ1-1 and M18.2b.

For biomass determination, Whatman™ filter discs (P1) were dried at 60° C. until their weight remained constant for 24 h, cooled to room temperature and weighed. Culture broths of strains MSEQ1SEQ5-1, MSEQ1-1 and M18.2b were filtered using those dried filter discs and the mycelia were washed with at least ten times the broth's volume of deionized water. Then the filter discs with the mycelia were dried at 60° C. until their weight remained constant for 24 h. The filter discs with the dried mycelia were weighed. The biomass concentration in the culture broth was then calculated by subtracting the mass of the dried filter disc from the mass of the dried filter disc with the mycelia and then dividing that value by the volume of the culture broth that had been filtered. The results of the measurements are shown in FIG. 6. Values are given in relation to the average biomass concentration in the culture broth of strain M18.2b which is set to 1. It is obvious from these data that strains MSEQ1SEQ5-1 produces significantly less biomass than strains MSEQ1-1 and M18.2b.

The viscosity of the culture broths of strains MSEQ1SEQ5-1 and MSEQ1-1 and M18.2b was measured using a Malvern Kinexus Lab+ KNX2110 rotational rheometer with the Vane tool (4Vnn:CUPnn) according to the manufacturer's instructions. The measurements were taken at a temperature of 20° C. and at a rotation velocity of 18.11 RPM (“rotations per minute”). The viscosities are depicted in FIG. 7 and are presented in relation to the viscosity of the culture broth of strain M18.2b, which is set to 1. It is obvious from these data that the viscosity of the culture broth produced with MSEQ1MSEQ5-1 is significantly lower than that of strains MSEQ1-1 and M18.2b.

SDS-PAGE analysis of the centrifuged culture supernatants of strains MSEQ1SEQ5-1, MSEQ1-1 and M18.2b was done using methods known to those skilled in the art (e.g. described by Jansohn et al. (Gentechnische Methoden, Elsevier, München)) and the Criterion XT system (BioRad). Equal volumes of culture supernatants were loaded in each lane. Precision Plus Protein™ All Blue Standards (BioRad) was used as protein size reference. The gel image is shown in FIG. 8. A person skilled in the art will recognize that—except the visibly increased protein concentration in the MSEQ1SEQ5-1- and MSEQ1-1 supernatants—the protein patterns of strains MSEQ1SEQ5-1, MSEQ1-1 and M18.2b are indistinguishable.

### SUMMARY

Taken together these data demonstrate that the disruption of the SEQ1 gene results in a significantly more efficient protein production, with more protein and less biomass being formed. The analysis of the secreted proteins by SDS-PAGE shows that their composition doesn't change significantly, indicating a general increase in protein production. In addition, the biomass production and the viscosity of the culture broth are significantly reduced as well.

### SEQUENCE LISTING

SEQ1 native gene
SEQ ID NO: 1
ATGGTTTCTGGCGACTACGCCTTCAACCCCGATCAACACGGCGCATATGCCGAACCGTACCAACAGCCGGACGACGGCCGGA
CTAGGACGCTGCTTGACAACCAAGCCTTCTTTTCTGACTTCGCGGGCCAGCAGCACTACGAACAGAACCAGATGGGCGACTA
TGGTGGCCCTAGATACTCCGGCGATGCCTTCTCTCCGACAGCAGCCATGGCTCCTCCGATGCTCACTGCCAACGACATGCCT
CCACCCGAGATATTGGAGTACCAGGCTCCGCTCGAGCCAAGAGAGGTCCCCTTTGCCATTCAGGATCCCCACGACAACAACA
CGGCCATGTCTTCGTTCGACAACATGGCTGCGGTACTCCGTCACCGTGCCCGCACCACTGCCAAAAGACCTGCATATTGGGT
CCTGGACAGCAAGGGCAAGGAGGTGGCATCCATTACATGGGACAAGCTGGCGTCAAGAGCGGAGAAAGTTGCACAAGTCATC
CGAGACAAAAGTCCTCTTTACCGTGGCGATCGAGTTGCCTTGATCTATCGTGATAGCGAAATCATTGACTTCGCCATTGCCT
TGCTGGGTTGCTTCATTGCTGGAGTTGTGGCTGTCCCGATCAATGACTTGCAAGACTACCAGCGCCTCAACTATATTCTCAC
CTCGACTCAGGCGCATCTGGCTCTTACTACCGAAAACAACCTCAAGACCTTCCAGAGAGACATTACTGCGCAGAAGCTCACG
TGGCCTAAAGGGGTCGAGTGGTGGAAGACCAACGAGTTCGGCGGTTACCATCCGAAGAAGAAGGAAGACGCACCTCCGTTAA
CTGTTCCCGACCTGGCCTATATTGAGTTTTCGCGAGCACCAACCGGCGACTTGAGGGGCGTTGTTCTCAGCCACAGGACAAT
CATGCACCAGATGGCCTGCCTCAGTGCCATAATCTCTACCGTTCCCACCAACGGCCCCGGCGATACCTTCAACTCGACGTTG
CGGGACAAGAACGGAAAGCTCATCGGCGGCGGAGCCAGCAGCGAGATATTGCTCTCCTATCTGGATCCCCGACAGGGCGTGG
GCATGATTCTCGGCGTTTTGCTGACCGTTTACGGCGGCCACACTACCGTCTGGTTCGATCACAAAGCCGTCGAGTCGCCTGG
CTTATACGCGCATCTGATTACCAAGTACAGAGCGACGATTATGATTGCGGATTACCCCGGGTTGAAGCGAGCTGCCTACAAC
TACCAGCAAGACCCCATGACGACACGAAACTTCAAAAAGGGGATGGAACCCAACTTCCAAGCGGTGAAGCTGTGCTTGATTG
ATACCCTGACCATTGATAGCGAGTTCCATGAAGTTCTGGCCGATAGATGGCTGCGGCCCCTGCGAAATCCGCGAGCGCGCGA
GGTCGTGGCGCCGATGCTCTGCCTCCCCGAGCATGGCGGCATGATCATTAGCGTTCGAGACTGGCTCGGCGGTGAAGAACGA
CTGGGAGTTCCGCTGAAACTGGACGAGTCTGACAGGGAGTCGGATGACGAGAAAGAAGAGGAAGAGAAGCCGGCCCCGTCAA
ACGGATTTGGTAGCTTGCTTGGTGGTGGAGCAGCGACAACCAAGGAGCAGGACGAGAAGATTGAGTTGGGCGAGGTTATCCT
TGACCGAGAGGCTCTCAAGACCAACGAGGTTGTCGTCTTGGCTCATGGCAACGAAGCTAGGAAGAAGACGTCGCTGGAGCCC
ACCATGGTCCGGGTCGGCGCCTTTGGATACCCTATCCCAGATGCCACGCTTGCTGTTGTGGACCCTGAGACTGGCCTCCTGG
CAGCGCCGCACACGATTGGCGAGATCTGGGTTGACTCTCCGTCTCTCTCTGGAGGCTTCTGGGCGCAGCCAAAGAACACCGA
GCTCATCTTCCACGCGCGTCCGTACAAGTTCGAGCCTGGCGAGCCGACGCCAACTGCCGTGGAGCCGGAATTCCTGCGAACC
GGCCTGCTTGGCACAGTCATCGAGGGCAAGATCTATGTGCTAGGATTGTACGAGGATCGGATACGACAAAAGGTCGAATGGG
TTGAGCACGGCCACGCGGGTATCGCCGAGTATCGCTACTTCTTTGTTCAGCACATTGTGGTGAGCATCGTCAAGAATGTCCC
CAAGATCCACGACTGCTCTGCCTTTGACGTCTTTGTCAATGACGAGCACTTGCCTGTCGTGGTCCTCGAGTCTGCCGCAGCA
TCAACGGCGCCTCTCACTTCGGGCGGCCCCCCTGTCCAGCCTGACACGGTTCTGTTAGACTCGCTGGCGGAGAAATGCATGG
AGGTGCTCATGCAGGAGCACCATCTTCGGGTTTACTGCGTTATGATCACAGCCCCGAACGCACTGCCGCGAGTGATCAAGAA
CGGAAGACGGGAAATAGGGAACATGCTCTGCCGGCGCGAGTTTGACCTTGGCAACCTCCCATGCGTGCATGTCAAATTTGGC
GTCGAACATGCGGTTCTCAACCTCCCGATTGGCGTTGACCCCATTGGTGGTATCTGGTCACCAATCGCCTCGGACTCGAGAA
TCAATATCCTGGCTCCCGCCGATAAGCAGTATTCTGGAATCGACCGCAGAGAGGTTGTTATGGACGACCGGACGTCTACACC
GCTCAACAATTTCAAGACCATCACCGATCTGATCCAGTGGCGTGTTGCTCGCCAGCCAGAGGAGCTCGCTTATTGTACCATT
GACGGCAGGGGCAGAGAGGGCAAGGGGATTCCGTGGAAGAAGTTTGACTCCAAGGTGGCGGCTGTGGCCATGTATCTGAAGA
ACAAAGTCAAGGTGCGGCCGGGCGACCACCTGGTCCTCATGTACACCCACTCCGAGGAGTTTGTCTTTGCCGTCCACGCGGG
AATCAACCTTGGCGCAGTCATTATTCCCATGGCGCCGCTTGACCAGAACCGGCTCAACGAAGATGTCCCTGCTTTCCTGCAC
CTGATCGCTGACTACAAGGTTAAGGCGGTCCTGGTCAACCAGGAAGTGGACCATTTGCTGAAGCTCAAGATCGTGTCGAGCC
ACATCAAACAGTCCGCACAGATCCTGAAGATCTCGATGCCGAATACCTACAACACTTCGAAGCCACCTAAGCAGAACAACGG
TCTTCGCGAGCTTGGGCTGACGATAGATCCCGCCTGGATCAGGCCTGGATACCCCGTCCTCATCTGGACATACTGGACGCCG
GACCAACGGAGAATCGCCGTCCAGCTGGGGCATGATACCATCCTGGGCATGTGCAAAGTGCAGAAGGAGACTTGTCAGATGA
CGAGCTTCCAGCCCGTTCTCGGTTGCGTAAGAAGCACAACGGGACTTGGTTTCGTGCACACGTGCCTGATGGGCATCTACGT
TGGCACCGCCACCTACCTGCTGTCTCCTGTCGAGTTCGCCCAAAATCCCATCTCTCTCTTTGTTACGTTGTCGAGGTACAAG
ATCAAGGACACCTATGCAACGCCGCAGATGCTTGACCATGCCATGTCGTCGATGCAGGCCAAGGGCTTTACAATGCACGAAC
TGAAGAATATGATGATTACTGCAGAGGGCCGGCCGCGGGTAGATGTATTCCAGAAGGTACGGATGCATTTTGCGAGCGCCGG
GCTGGATAGGACGGCCATCAACACGGTCTACTCGCATGTGCTCAACCCGATGATTGCTTCGAGGTCTTACATGTGCATCGAG
CCTATTGAGCTCTGGCTCGACACCAAGGCTCTTCGACGCGGCCTCGTCGTCCCGGTCGATCACGATTCAAACCCGCAAGCTC
TTCTCCTGCAGGATTCCGGCATGGTGCCGGTGTCTACCCAGATTGCCATTGTCAACCCCGAGAGCCGCGCGCATTGCTACGA
TGGAGAATATGGCGAGATCTGGGTCGACTCCGAGGCGTGCGTAAAGGCCTTTTACGGCTCCAAGGAAGCGTTTGACGTGGAG
CGCTTCGACGGCCGGACGGTCGACGGCGACCCCAACGTGCGATACGTGCGAACTGGTGACTTGGGCTTTTTGTATAATGTCA
ACCGGCCTATCGGGCCCAACGGCGCCCTGGTGGAGATGCAAGTCTTGTTTGTGCTCGGTAGCATCGGCGAGACTTTTGAAAT
TAACGGTTTGAGTCACTTCCCCATGGATATTGAGCTGTCGGTGGAACGCTGCCACCGCAACATTGTACCCAACGGCTGGTAA
GTACAGGGCCAACTCTTCTGTGAGATGCTACTTGACTAATAGTTGGTGATGTGCAGTGCTGTATTCCAAGCTGGTGGCTTGG
TCGTGGTCCTGGTAGAGGTGAGCCGCAAGTCTTACCTCGCCTCCATGGTGCCAGTCATTGTCAACGCCATCTTGAGCGAGCA
TCAGATCGTTGCCGATATTGTGGCCTTTGTCAACAAGGGCGACTTCCCACGCTCTCGCCTGGGAGAGAAGCAGCGAGGAAAG
ATCCTCGCCGGATGGGTTACGCGCAAGATGCGCACCATGGCCCAGTTCGCCATCAGAGATCTCGACGCCAGCATGCTCGAGC
CGGGTGAGGGTCCGGATGCCAATAGGACCTCTACGGGTAGCCTCCGTAGCCTGGGCGCCGCCGTCCCGCCAAACTTCAAGAT
GGTTGGACAGGCGCCGCAGATACTGGAACAGGAGGAATTTACGCAGCAGATGGATCACATGGCCCATTCGGAGCCGGTCAGG
CATGCCATGGCCGCTCCAGAGGAGCAGCAGGCGCCGGCGGCCTATTACGCTGGCGGCCAGGAGGCTGCTTTCATGCAGGGCT
ATAATCAGCAGCCGCCACCACCACCACCAAGCAGCCAGGGAGGATACCAGTACGAGCAATTCGAGCCAGTGCAAGCACAGCA
ACAGTACCAGCCGCAGTCACAGCATCAGTTTGAACCATCTCAAGCCTTTGAGCCAGCGCAGCAATACGAGCCAGCGCAACAG
CACGAGCAGGAGCCAAGGCCGATGGACTCTCGAGGACAAGATGCGCCGTCCATTGTTGAGCCAGAGACCTCAGCCTCCGTGC
CTGATACGCCGCCGCCGAGAAACAGGTTGAGTCAAGGGCCGCCCCAGATCCGGCTCCCGGGCGTTGACGGACGGGAAGGGCT
CGACTTCTGGGGAGGCAACGACGAGACGGACTGGACGGCCGATGCCATGATGCACATGAATCTCACTGGCCCACGGTAA
Hygromycin B resistance marker
SEQ ID NO: 2
TGCAAGGCGATTAAGTTGGGTAACGCCAGGGTTTTCCCAGTCACGACGTTGTAAAACGACGGCCAGTGAGCGCGACGTAATA
CGACTCACTATAGGGCGAATTGGCGGAAGGCCGTCAAGGCCTAGGCGCGCCATGAGCTCGTTAACAAGACACAGCCCTATAA
CTTCGTATAATGTATGCTATACGAAGTTATATAACGGTGAGACTAGCGGCCGGTCCCCTTATCCCAGCTGTTCCACGTTGGC
CTGCCCCTCAGTTAGCGCTCAACTCAATGCCCCTCACTGGCGAGGCGAGGGCAAGGATGGAGGGGCAGCATCGCCTGAGTTG
GAGCAAAGCGGCCCGGCCGCCATGGGAGCAGCGAACCAACGGAGGGATGCCGTGCTTTGTCGTGGCTGCTGTGGCCAATCCG
GGCCCTTGGTTGGCTCACAGAGCGTTGCTGTGAGACCATGAGCTATTATTGCTAGGTACAGTATAGAGAGAGGAGAGAGAGA
GAGAGAGAGAGAGAGGGGAAAAAAGGTGAGGTTGAAGTGAGAAAAAAAAAAAAAAAAAAAAATCCAACCACTGACGGCTGCC
GGCTCTGCCACCCCCCTCCCTCCACCCCAGACCACCTGCACACTCAGCGCGCAGCATCACCTAATCTTGGCTCGCCTTCCCG
CAGCTCAGGTTGTTTTTTTTTTCTCTCTCCCTCGTCGAAGCCGCCCTTGTTCCCTTATTTATTTCCCTCTCCATCCTTGTCT
GCCTTTGGTCCATCTGCCCCTTTGTCTGCATCTCTTTTGCACGCATCGCCTTATCGTCGTCTCTTTTTTCACTCACGGGAGC
TTGACGAAGACCTGACTCGTGAGCCTCACCTGCTGATTTCTCTCCCCCCCTCCCGACCGGCTTGACTTTTGTTTCTCCTCCA
GTACCTTATCGCGAAGCCGGAAGAACCTCTTAACCTCTAGATGAAAAAGCCTGAACTCACCGCCACGTCTGTCGAGAAGTTC
CTGATCGAAAAGTTCGACAGCGTCTCCGACCTGATGCAGCTCTCGGAGGGCGAAGAATCTCGTGCTTTCAGCTTCGATGTAG
GAGGGCGTGGATATGTCCTGCGGGTAAATAGCTGCGCCGATGGTTTCTACAAAGATCGTTATGTTTATCGGCACTTTGCATC
GGCCGCGCTCCCGATTCCGGAAGTGCTTGACATTGGGGAATTCAGCGAGAGCCTGACCTATTGCATCTCCCGCCGTGCACAG
GGTGTCACGTTGCAAGACCTGCCTGAAACCGAACTGCCCGCTGTTCTGCAGCCGGTCGCGGAGGCCATGGATGCGATCGCTG
CGGCCGATCTCAGCCAGACGAGCGGGTTCGGCCCATTCGGACCGCAAGGAATCGGTCAATACACTACATGGCGTGATTTCAT
ATGCGCGATTGCTGATCCCCATGTGTATCACTGGCAAACTGTGATGGACGACACCGTCAGTGCGTCCGTCGCGCAGGCTCTC
GATGAGCTGATGCTTTGGGCCGAGGACTGCCCCGAAGTCCGGCACCTCGTGCACGCGGATTTCGGCTCCAACAATGTCCTGA
CGGACAATGGCCGCATAACAGCGGTCATTGACTGGAGCGAGGCGATGTTCGGGGATTCCCAATACGAGGTCGCCAACATCTT
CTTCTGGAGGCCGTGGTTGGCTTGTATGGAGCAGCAGACGCGCTACTTCGAGCGGAGGCACCCGGAGCTTGCAGGATCGCCG
CGGCTCCGGGCGTATATGCTCCGCATTGGTCTTGACCAACTCTATCAGAGCTTGGTTGACGGCAATTTCGATGATGCAGCTT
GGGCGCAGGGTCGATGCGACGCAATCGTCCGATCCGGAGCCGGGACTGTCGGGCGTACACAAATCGCCCGCAGAAGCGCGGC
CGTCTGGACCGATGGCTGTGTAGAAGTACTCGCCGATAGTGGAAACCGACGCCCCAGCACTCGTCCGAGGGCAAAGGAATAG
ATGCATGGCTTTCGTGACCGGGCTTCAAACAATGATGTGCGATGGTGTGGTTCCCGGTTGGCGGAGTCTTTGTCTACTTTGG
TTGTCTGTCGCAGGTCGGTAGACCGCAAATGAGCAACTGATGGATTGTTGCCAGCGATACTATAATTCACATGGATGGTCTT
TGTCGATCAGTAGCTAGTGAGAGAGAGAGAACATCTATCCACAATGTCGAGTGTCTATTAGACATACTCCGAGAATAAAGTC
AACTGTGTCTGTGATCTAAAGATCGATTCGGCAGTCGAGTAGCGTATAACAACTCCGAGTACCAGCGAAAGCACGTCGTGAC
AGGAGCAGGGCTTTGCCAACTGCGCAACCTTGCTTGAATGAGGATACACGGGGTGCAACATGGCTGTACTGATCCATCGCAA
CCAAAATTTCTGTTTATAGATCAAGCTGGTAGATTCCAATTACTCCACCTCTTGCGCTTCTCCATGACATGTAAGTGCACGT
GGAAACCATACCCAATATAACTTCGTATAATGTATGCTATACGAAGTTATAGGGCTCTTGTCTGTT
LIC reception vector
SEQ ID NO: 3
TTTATCAGGGTTATTGTCTCATGAGCGGATACATATTTGAATGTATTTAGAAAAATAAACAAATAGGGGTTCCGCGCACATT
TCCCCGAAAAGTGCCACCTGACTAAACCAGACAGACAGCTGTCTCTCCTCTCTAACATGTGAGCAAAAGGCCAGCAAAAGGC
CAGGAACCGTAAAAAGGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCATAGCTCACGCTGTAGGTA
TCTCAGTTCGGTGTAGGTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTACACTAGAAGGACAGTATTTGGTATCTG
CGCTCTGCTGAAGCCAGTTACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTGTCACGCTCGTCGTTTGGTATGGCTT
CATTCAGCTCCGGTTCCCAACGATCAAGGCGAGTTACATGATCCCCCATGTTGTGCAAAAAAGCGGTTAGCTCCTTCGGTCC
TCCGATCGTTGTCAGAAGTAAGTTGGCCGCAGTGTTATCACTCATGGTTATGGCAGCACTGCATAATTCTCTTACTGTCATG
CCATCCGTAAGATGCTTTTCTGTGACTGGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAGTGCTCATCATTGGAAAACGTTCTTCGGG
GCGAAAACTCTCAAGGATCTTACCGCTGTTGAGATCCAGTTCGATGTAACCCACTCGTGCACCCAACTGATCTTCAGCATCT
TTTACTTTCACCAGCGTTTCTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCA
pSEQ1flank5
SEQ ID NO: 4
CTAAATTGTAAGCGTTAATATTTTGTTAAAATTCGCGTTAAATTTTTGTTAAATCAGCTCATTTTTTAACCAATAGGCCGAA
ATCGGCAAAATCCCTTATAAATCAAAAGAATAGACCGAGATAGGGTTGAGTGGCCGCTACAGGGCGCTCCCATTCGCCATTC
AGGCTGCGCAACTGTTGGGAAGGGCGTTTCGGTGCGGGCCTCTTCGCTATTACGCCAGCTGGCGAAAGGGGGATGTGCTGCA
AGGCGATTAAGTTGGGTAACGCCAGGGTTTTCCCAGTCACGACGTTGTAAAACGACGGCCAGTGAGCGCGACGTAATACGAC
TCACTATAGGGCGAATTGGCGGAAGGCCGTCAAGGCCGCATGACTGAATAACTTCGGGCGGCCCCCCTGTCCAGCCTGACAC
GGTTCTGTTAGACTCGCTGGCGGAGAAATGCATGGAGGTGCTCATGCAGGAGCACCATCTTCGGGTTTACTGCGTTATGATC
ACAGCCCCGAACGCACTGCCGCGAGTGATCAAGAACGGAAGACGGGAAATAGGGAACATGCTCTGCCGGCGCGAGTTTGACC
TTGGCAACCTCCCATGCGTGCATGTCAAATTTGGCGTCGAACATGCGGTTCTCAACCTCCCGATTGGCGTTGACCCCATTGG
TGGTATCTGGTCACCAATCGCCTCGGACTCGAGAATCAATATCCTGGCTCCCGCCGATAAGCAGTATTCTGGAATCGACCGC
AGAGAGGTTGTTATGGACGACCGGACGTCTACACCGCTCAACAATTTCAAGACCATCACCGATCTGATCCAGTGGCGTGTTG
CTCGCCAGCCAGAGGAGCTCGCTTATTGTACCATTGACGGCAGGGGCAGAGAGGGCAAGGGGATTCCGTGGAAGAAGTTTGA
CTCCAAGGTGGCGGCTGTGGCCATGTATCTGAAGAACAAAGTCAAGGTGCGGCCGGGCGACCACCTGGTCCTCATGTACACC
CACTCCGAGGAGTTTGTCTTTGCCGTCCACGCGGGAATCAACCTTGGCGCAGTCATTATTCCCATGGCGCCGCTTGACCAGA
ACCGGCTCAACGAAGATGTCCCTGCTTTCCTGCACCTGATCGCTGACTACAAGGTTAAGGCGGTCCTGGTCAACCAGGAAGT
GGACCATTTGCTGAAGCTCAAGATCGTGTCGAGCCACATCAAACAGTCCGCACAGATCCTGAAGATCTCGATGCCGAATACC
TACAACACTTCGAAGCCACCTAAGCAGAACAACGGTCTTCGCGAGCTTGGGCTGACGATAGATCCCGCCTGGATCAGGCCTG
GATACCCCGTCCTCATCTGGACATACTGGACGCCGGACCAACGGAGAATCGCCGTCCAGCTGGGGCATGATACCATCCTGGG
CATGTGCAAAGTGCAGAAGGAGACTTGTCAGATGACGAGCTTCCAGCCCGTTCTCGGTTGCGTAAGAAGCACAACGGGACTT
GGTTTCGTGCACACGTGCCTGATGGGCATCTACGTTGGCACCGCCACCTACCTGCTGTCTCCTGTCGAGTTCGCCCAAAATC
CCATCTCTCTCTTTGTTACGTTGTCGAGGTACAAGATCAAGGACACCTATGCAACGCCGCAGATGCTTGACCATGCCATGTC
GTCGATGCAGGCCAAGGGCTTTACAATGCACGAACTGAAGAATATGATGATTACTGCAGAGGGCCGGCCGCGGGTAGATGTA
TTCCAGAAGGTACGGATGCATTTTGCGAGCGCCGGGCTGGATAGGACGGCCATCAACACGGTCTACTCGCATGTGCTCAACC
CGATGATTGCTTCGAGGTCTTACATGTGCATCGAGCCTATTGAGCTCTGGCTCGACACCAAGGCTCTTCGACGCGGCCTCGT
CGTCCCGGTCGATCACGATTCAAACCCGCAAGCTCTTCTCCTGCAGGATTCCGGCATGGTGCCGGTGTCTACCCAGATTGCC
ATTGTCAACCCCGAGAGCCGCGCGCATTGCTACGATGGAGAATATGGCGAGATCTGGGTCGACTCCGAGGCGTGCGTAAAGG
CCTTTTACGGCTCCAAGGAAGCGTTTGACGTGGAGCGCTTCGACGGCCGGACGGTCGACGGCGACCCCAACGTGCGATACGT
GCGAACTGGTGACTTGGGCTTTTTGTATAATGTCAACCGGCCTATCGGGCCCAACGGCGCCCTGGTGGAGATGCAACTTGTT
TGTGCTCGGTAGCATCGGCGAGACTTTTGAAATTAACGGTTTGAGTCACTTCCCCATGGATATTGAGCTGTCGGTGGAACGC
TGCCACCGCAACATTGTACCCAACGGCTGGTAAGTACAGGGCCAACTCTTCTGTGAGATGCTACTTGACTAATAGTTGGTGA
TGTGCAGTGCTGTATTCCAAGCTGGTGGCTTGGTCGTGGTCCTGGTAGAGGTGATAACAAGACACAGCCCGGGCTCTTGTCT
GTTACTGGGCCTCATGGGCCTTCCGCTCACTGCCCGCTTTCCAGTCGGGAAACCTGTCGTGCCAGCTGCATTAACATGGTCA
TAGCTGTTTCCTTGCGTATTGGGCGCTCTCCGCTTCCTCGCTCACTGACTCGCTGCGCTCGGTCGTTCGGGTAAAGCCTGGG
GTGCCTAATGAGCAAAAGGCCAGCAAAAGGCCAGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTGCCTGACTCCCCGTC
GTGTAGATAACTACGATACGGGAGGGCTTACCATCTGGCCCCAGTGCTGCAATGATACCGCGAGAACCACGCTCACCGGCTC
CAGATTTATCAGCAATAAACCAGCCAGCCGGAAGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATCAAGGCGAGTTACATGATCCCCCATGT
TGTGCAAAAAAGCGGTTAGCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGCTGTTGAGATCCAGTTCGATGTAACC
CACTCGTGCACCCAACTGATCTTCAGCATCTTTTACTTTCACCAGCGTTTCTGGGTGAGCAAAAACAGGAAGGCAAAATGCC
GCAAAAAAGGGAATAAGGGCGACACGGAAATGTTGAATACTCATACTCTTCCTTTTTCAATATTATTGAAGCATTTATCAGG
GTTATTGTCTCATGAGCGGATACATATTTGAATGTATTTAGAAAAATAAACAAATAGGGGTTCCGCGCACATTTCCCCGAAA
AGTGCCAC
SEQ5 native gene
SEQ ID NO: 5
ATGAGGGCCTATCAGATCGAGATGCTCGACAAGAGCCTCAAGCAAAATGTCATTGTTGCTGTATGTTGAAGTTTCTCTCCAA
TCCCCCGTCTCCCCCTTTGCTGTCGTTGTCTTCGACGTTGAAAGACATGTCCATTGACCAAGGGGCGTTGTTATAAATCTAG
ATGGACACGGGAAGTGGCAAGACTCAAGTGTAAGTTGTGCATCTTCATCATCGGCAGCCCACGTAACCTGTGCCAGCCCTTA
GCACCCTTCTTCGCAAAAGACTGACTTGGCGCTTGCATCAGAGCTGTGCTTCGTATCAAGAAGGAGCTGGAAATCTGCGATG
CATCAAAGGTGAGTCTGCCGTCTGGATACAGTTGCACAACGACCTGGACAGCTGCACTGACGCAGCACGCATCAGATCATCT
GGTTCATCGCGCCAACAGTTTCGCTGTGTCATCAGCAACACGATGTGCTCAAGTTGCAGATACCTGCCGTGCCCATGATGAC
ACTGGCCGGGAACTCCAATATCGATGCTTGGGGGCCGGATATCTGGGCCATTCTTCTCGACACGGTTCGAATTGTCATATCC
ACACCCCAGGTTCTGCTCGATGCCCTTGACCATGCTTACCTGAACTTGGGTCTTCTGGCGCTGCTTGTATTTGATGAAGGTA
TGGGACGACCTGCCTTCACTCTGTAAAGGCAAAGGGGCCGCCAGAAGTTGCAAATCGCTGACGTGTCTTGTGCAAAAGTCCA
CAACTGCATTGGCAGAAGTCCAGGCGGCAAAATCATGCTCCACCACTACCATCCGCGCAAGCTGGCTGGTGAAAGCGTGCCT
GCTGTTCTGGGTCTGACGGCAACTCCGAGCATTCAGTCTGAGCTTGCCGATATTGATGCCTTGGAATGGCTGATGGATGCAA
GATGCGTCTCGCCCACTCTCCATCGCGACGAACTGCTCAAATGCGTCAAGAGGCCCAATATCAAGCACATCATCTATAAAGC
CGGCAAAGAAGACATCACGACGCCCACCATGCGCGACTTGGATCGGGTCTACCGGGCGCTGGACATTCTCGAAGACCCCTAC
ATACTCATGCTGCGCAACAACCCTACGGACCGAAACAACCGCCTGCTGCTAACAGCCATTGAAAAGTACGATACCTACACAC
AGAACCAGATGAAGTCGTTCTGCGCCCGATCAAGAGAGATATGCAAGCAACTCGGTCCCTGGGCTGCTGACCTCTTCATCTG
GAAGGCCATCTCAGCTCACTTGGACAAGGTGGACAGGCAGACGGATGGAGTTGACGAGTATGGCAACAAGTGGTCGTCGGGG
TCGACAAGCTTCCTGGAAAAGAAGCACCTGGCCGACATCTATCGTCGAGTCAAGGTCCAACGTCCTTCCGATGTGCCACAGG
TCTTTGAAGACATTTCCGACAAGGTCGGTAAGCTAATCTTTGAGCTTCTGTCGGTAGAGGAGCCCACGGTGGGCATCATCTT
CGTCGAGGAACGAGTCATGGTTGCTATGCTGGCCGAGGTTCTCTCTGTCAACCACACAATCACGTCCCGGTACCGGATCGGG
ACCATGGTTGGCACCTCAAATTACGCTGGGCGGCGGAAGGCCGTTTATGACTTCGACCAGAAAACGGACTACAAGGACCTGC
AGAGCTTCCGCTCCGGCAAGATTAACCTGCTGATTGCGACGTCAGTGCTGGAGGAGGGCATCGACGTGCCTGCCTGCAACCT
AGTCATATGCTTTGACACTCCGACGACCCCAAAGTCCTTTATCCAGCGGCGCGGACGGGCTCGCTCCAAGGACTCGAATCTC
CTTCTTTTCTTTGACGATGCCAACCCTGCGATCTTGAAGTGGCAGGCGAAAGAGGAGGAGATGAACAGGATCTTCGAAGACG
AAGAGAGGGCGATTCGCGAACTCGGCAAACTGGAAGATTCGGAGAGTCCGAGCACCATCTCCTTCACCGTCCCGTCTACCGG
CGCAAGGCTAGATTTTGACAATGCGAAGCAGCACCTCGAGCACTTCTGCAGAGTCTTGTGCCCGTCGGACTTTGTGGACAGC
CGCCCGGACTACATCATCCGCAGGGAGCAGGACTCTCCTTTGTTGACTGCCATTGTACTGCTCCCTCCGTTTCTGCCGGTGA
ATCTGAGGCAGCACACCAGTGCTTCTCCTTGGCGCTCCGAGAAGAACGCCACCAAGGATGCTGCGTATCAGGCGTATATAGC
CCTGTATGACGCGAAGCTCGTCAACGAGAACCTGCTGCCCTTCAAGTCCAGCGACATGCTCGGAATCGATAAGCGAGTATCC
GAGGTGCCGGTCGAGCCGTTGATGAAGCCATGGCATCGTGTCGCTCCTGCGTGGCGGGAAGCTGGCGACAAGTGGCTTTACT
CCTTGAGCTGCGTGGAGGAGGACGGCCGAGTAAGTGCAGAGTACGAGGTTCTGCTGCCAGTCTGGCTGAACCAGCCTCAGCC
CCTGAAAATGTTCCTCGACCGCAATCACCAGGTGGAGTTGCAGCTGAAGGCCGGGATACCCGTGCCGCACGAGCAAGTTGCG
TCCCTGCCAGATCATACATCGACTTTGCTGGCGCTGCATTTCGGTCATCGATGGCCTCTCGAGCAGAAAGAGCACGTCATTC
GGGTCTGGGCCAAGGATCAACCCCTATCGCTGAACCAAATTGGCGAGCTCACATACGATCCACAGAATGAGAGCGTCAGCCG
GGGAGAGTTTCTCATCCGGGACAACACCAGAGCCCCCTACCTGTACAAGGATACCATTGCGTTCAAGCCCGAACCGAGCCAG
GTCCAGAATACCTTTTACGAGTACGACAAGGCGCCCGAAGACGTGCCGTATCTCGTGCTCACCAAATGGACGCGGCGGACCG
ACTTTCTGCATCGCCTCCAAGGGAATCCCGCCAAGAATGAGGTTAGTAGCAAGCCATACGCACGCGTATATCCGCTGTCGTG
GGCGACAGTCGATACCATCCCCGCCAGGCACGCCCAGTTTGGCATGCTGATCCCGACCATGATCCACGAGCTCGGCGTCATG
CTCATGGCCAAGGAGCTGGCCTACTCCGTTCTCGACGAGGTTGGCATTTCGGATCTGCAGCTGGTCAAGGAGGCCATCAGCG
CGCGGAGTGCCTCGGAGCCGGTGAATTACGAGAGGCTGGAGTTTTTGGGCGACTCGATTCTCAAGTTTTGTGCCTGTATGCG
CGCCGCTGCTGAAAGTAAGTTGCTCAAGCGTTTTACTCATATATGACTCCTGTGTGCACCTGTCCTCTGACATGGAACTGTT
TTGCTGACCACATTTGATACTGCCTAGAACCCGACTATCCCGAGGGCTATCTCTCGTATTGGAGAGACCGACTCGTCTCCAA
CTCGAGGCTGTACAAAGCCGCTCTCGAGTTTGGGCTGCCGAGGTTCATCTTGACGAAACCTTTTACCGGTCAAAAGTGGCGC
CCACTCTACCTGGACGAGGTCCTCCAGCAAGGGGACGTCGCTACGCCGGAGAAGAGAAAATTATCGACCAAGACGCTCGCAG
ACGTGGTCGAGGCGCTGATCGGGGCCTCATACGTCGATGGAGGCCTTTCAAAGGCAGTGACTTGCATCTCAAAATTCGTCCC
CGAAGGCTCGTGGACCAGTGTTGATGCAGATAGAGAGTCTCTCTTTGCGAGAGTGCCAGACGGCGAGCCTCTCCCGCCGCCA
TTGGAGCCGCTGGAGAAGTTGATCGGCTACACGTTCCAGAAAAAGGCGCTCTTGATGGAGGCTCTGACGCATGCCTCGTATG
CTGCAGACTTCGGAACGCGATCTCTCGAGAGGCTCGAATTCATAGGAGACGCTGTCCTGGACAACATTATCGTTACGAAGCT
CTTTAGGCTGAAGCCAGCGCTGCCCCATTTCAGGATGCATACGCTGAAGACGGGCCTGGTGAATGGGGACTTTCTTGCTTTC
ATGACAATGGAGCACGGAGTGCAACTGGCGGCGGACCCTGTGGTGACAGAAGAAGCTACGGTGGAGGTCCCGGAAACGATTT
CCTACCTGTGGTCGTTTTTGAGGCAGGCCTCTTTTCCCATTGCCATCGAGCTGAAGGAGACGAACAAGCGGCACGCTGCCCT
GAGAGAGCAGATTCACGAAGCAATGGACAATGACGATCATTACCCCTGGGCGCTGCTGGCCGCCCTGAGCCCGAAGAAGTTC
TACTCTGACCTCTTCGAGGCGGTTCTCGGCGCTGTGTGGATCGACTCCGGGTCGCTGGCGGCGTGCGAGGGCATGGTTGCGC
AGTTTGGGATCTTAAAGTACATGGATCGGCTGCTGCGTGACGAAGTCCACGTGCAGCATCCTAAGGAGGAGCTGGGCATGTG
GGCAAACACAGAGACTGTGACGTACGAGCTCGAGATGAAGGGGAGCGAGGAGAGCGCGGGGGAGAGGGAGTATTTCTGCAAG
GTGTTTGTTGGAAAGAGGGAGGTTGTGGAGGTTCGTGGGGGGGTCAATAAGGAGGAGGTGAAGACGAAGGGTGCGACGGAGG
CGTTGCGGATTTTGAGGGAGGAGAAAAGGCGCGGTGCTGAGGATGTGGTGATGGTGGGATAA
hygrfw
SEQ ID NO: 6
TGCAAGGCGATTAAGTTGGG
hygrrv
SEQ ID NO: 7
CGGCGAGGATCTTTCCTCGCTGCTTCTCTCAACAGACAAGAGCCCTATAACTTC
SEQ1fl3fw
SEQ ID NO: 8
TTGTCAACGCCATCTTGAGC
SEQ1fl3rv
SEQ ID NO: 9
ACCAACCAGTCCATCCTCTG
fus1
SEQ ID NO: 10
AAACCAGACAGACAGTATACGACTCACTATAGGGCG
fus2
SEQ ID NO: 11
GTTAACAGACAAGAGCCCGAAGTTATTCGGGTAGTAGAGTTTGAAAGGGG
fus3
SEQ ID NO: 12
AGAGAGGAGAGACAGTGTTAACAGACAAGAGCCCGAAG
SEQ1MKOfw
SEQ ID NO: 13
ATGTGCTAGGATTGTACGAG
SEQ1MKO1rv
SEQ ID NO: 14
ATAATAGCTCATGGTCTCAC
SEQ1MKO2rv
SEQ ID NO: 15
TTGACAAAGGCCACAATATC
M1Seq-01
SEQ ID NO: 16
ATCGCTACTTCTTTGTTCAG
M1Seq-02
SEQ ID NO: 17
CAGCTTGGAATACAGCACTG
SEQ5M5fw
SEQ ID NO: 18
GACTCTCTATCTGCATCAAC
SEQ5M5rv
SEQ ID NO: 19
TGACCTGGAAAGCTTTCAATGTAGAGGTAGACTAGTCAAAGAAGACATCACGAC
SEQ5M3fw
SEQ ID NO: 20
CGCATGGTGGGCGTCGTGATGTCTTCTTTGACTAGTCTACCTCTACATTGAAAG
SEQ5M3rv
SEQ ID NO: 21
GATTACCTGTCAAGTCTATG
SEQ5Mnestfw
SEQ ID NO: 22
GACAGTCCTGCAGGAGTCACTGCCTTTGAAAG
SEQ5Mnestrv
SEQ ID NO: 23
GACAGTCCTGCAGGTGTAAGGATAAAGGACGAC
LIC1fw
SEQ ID NO: 24
CTAGGAGTTCTGCCTTGGGTTTAAACGAGAGAAAGACTC
LIC1rv
SEQ ID NO: 25
CTAGGAGTCTTTCTCTCGTTTAAACCCAAGGCAGAACTC
amdS
SEQ ID NO: 26
GGATGTACGACGTATATCCATCTTTAACTAGTCATCATTGGATAGGCAGATTACTCAGCCTGAATGACATCAACATGTTACC
CATGATACAATAGGTCACACAAACAAGCGCTAAGATGCACTTGGTATGACAAGCCCAGTAGTCCGTTTCAAAAGACCTAGAT
GATGAACTACAACATGAGGTGTTGCCTCCTGATCCAGTCCAACTGCAAACGCTGATGTATACTCAATCAAGCCTGATGTAAA
TGCTGCGACTGCATTCGCTGGATATGAAGATCAAAGAGAGCTCTGATGGGTCCAATATAGCCGGGTTTTGTTAGGACAGTCC
ACCACACCGATATTAGAATTGGTCAAGCACCTTATCATTTCATAGAGATTGCGGTTTCTAGATCTACGCCAGGACCGAGCAA
GCCCAGATGAGAACCGACGCAGATTTCCTTGGCACCTGTTGCTTCAGCTGAATCCTGGCAATACGAGATACCTGCTTTGAAT
ATTTTGAATAGCTCGCCCGCTGGAGAGCATCCTGAATGCAAGTAACAACCGTAGAGGCTGACACGGCAGGTGTTGCTAGGGA
GCGTCGTGTTCTACAAGGCCAGACGTCTTCGCGGTTGATATATATGTATGTTTGACTGCAGGCTGCTCAGCGACGACAGTCA
AGTTCGCCCTCGCTGCTTGTGCAATAATCGCAGTGGGGAAGCCACACCGTGACTCCCATCTTTCAGTAAAGCTCTGTTGGTG
TTTATCAGCAATACACGTAATTTAAACTCGTTAGCATGGGGCTGATAGCTTAATTACCGTTTACCAGTGCCGCGGTTCTGCA
GCTTTCCTTGGCCCGTAAAATTCGGCGAAGCCAGCCAATCACCAGCTAGGCACCAGCTAAACCCTATAATTAGTCTCTTATC
AACACCATCCGCTCCCCCGGGATCAATGAGGAGAATGAGGGGGATGCGGGGCTAAAGAAGCCTACATAACCCTCATGCCAAC
TCCCAGTTTACACTCGTCGAGCCAACATCCTGACTATAAGCTAACACAGAATGCCTCAATCCTGGGAAGAACTGGCCGCTGA
TAAGCGCGCCCGCCTCGCAAAAACCATCCCTGATGAATGGAAAGTCCAGACGCTGCCTGCGGAAGACAGCGTTATTGATTTC
CCAAAGAAATCGGGGATCCTTTCAGAGGCCGAACTGAAGATCACAGAGGCCTCCGCTGCAGATCTTGTGTCCAAGCTGGCGG
CCGGAGAGTTGACCTCGGTGGAAGTTACGCTAGCATTCTGTAAACGGGCAGCAATCGCCCAGCAGTTAGTAGGGTCCCCTCT
ACCTCTCAGGGAGATGTAACAACGCCACCTTATGGGACTATCAAGCTGACGCTGGCTTCTGTGCAGACAAACTGCGCCCACG
AGTTCTTCCCTGACGCCGCTCTCGCGCAGGCAAGGGAACTCGATGAATACTACGCAAAGCACAAGAGACCCGTTGGTCCACT
CCATGGCCTCCCCATCTCTCTCAAAGACCAGCTTCGAGTCAAGGTACACCGTTGCCCCTAAGTCGTTAGATGTCCCTTTTTG
TCAGCTAACATATGCCACCAGGGCTACGAAACATCAATGGGCTACATCTCATGGCTAAACAAGTACGACGAAGGGGACTCGG
TTCTGACAACCATGCTCCGCAAAGCCGGTGCCGTCTTCTACGTCAAGACCTCTGTCCCGCAGACCCTGATGGTCTGCGAGAC
AGTCAACAACATCATCGGGCGCACCGTCAACCCACGCAACAAGAACTGGTCGTGCGGCGGCAGTTCTGGTGGTGAGGGTGCG
ATCGTTGGGATTCGTGGTGGCGTCATCGGTGTAGGAACGGATATCGGTGGCTCGATTCGAGTGCCGGCCGCGTTCAACTTCC
TGTACGGTCTAAGGCCGAGTCATGGGCGGCTGCCGTATGCAAAGATGGCGAACAGCATGGAGGGTCAGGAGACGGTGCACAG
CGTTGTCGGGCCGATTACGCACTCTGTTGAGGGTGAGTCCTTCGCCTCTTCCTTCTTTTCCTGCTCTATACCAGGCCTCCAC
TGTCCTCCTTTCTTGCTTTTTATACTATATACGAGACCGGCAGTCACTGATGAAGTATGTTAGACCTCCGCCTCTTCACCAA
ATCCGTCCTCGGTCAGGAGCCATGGAAATACGACTCCAAGGTCATCCCCATGCCCTGGCGCCAGTCCGAGTCGGACATTATT
GCCTCCAAGATCAAGAACGGCGGGCTCAATATCGGCTACTACAACTTCGACGGCAATGTCCTTCCACACCCTCCTATCCTGC
GCGGCGTGGAAACCACCGTCGCCGCACTCGCCAAAGCCGGTCACACCGTGACCCCGTGGACGCCATACAAGCACGATTTCGG
CCACGATCTCATCTCCCATATCTACGCGGCTGACGGCAGCGCCGACGTAATGCGCGATATCAGTGCATCCGGCGAGCCGGCG
ATTCCAAATATCAAAGACCTACTGAACCCGAACATCAAAGCTGTTAACATGAACGAGCTCTGGGACACGCATCTCCAGAAGT
GGAATTACCAGATGGAGTACCTTGAGAAATGGCGGGAGGCTGAAGAAAAGGCCGGGAAGGAACTGGACGCCATCATCGCGCC
GATTACGCCTACCGCTGCGGTACGGCATGACCAGTTCCGGTACTATGGGTATGCCTCTGTGATCAACCTGCTGGATTTCACG
AGCGTGGTTGTTCCGGTTACCTTTGCGGATAAGAACATCGATAAGAAGAATGAGAGTTTCAAGGCGGTTAGTGAGCTTGATG
CCCTCGTGCAGGAAGAGTATGATCCGGAGGCGTACCATGGGGCACCGGTTGCAGTGCAGGTTATCGGACGGAGACTCAGTGA
AGAGAGGACGTTGGCGATTGCAGAGGAAGTGGGGAAGTTGCTGGGAAATGTGGTGACTCCATAGCTAATAAGTGTCAGATAG
CAATTTGCACAAGAAATCAATACCAGCAACTGTAAATAAGCGCTGAAGTGACCATGCCATGCTACGAAAGAGCAGAAAAAAA
CCTGCCGTAGAACCGAAGAGATATGACACGCTTCCATCTCTCAAAGGAAGAATCCCTTCAGGGTTGCGTTTCCAGTCTAGAC
ACGTATAACGGCACAAGTGTCTCTCACCAAATGGGTTATATCTCAAATGTGATCTAAGGATGGAAAGCCCAGAATATTGGCT
GGGTTGATGGCTGCTTCGAGTGCAGTCTCATGCTGCCACAGGTGACTCTGGATGGCCCCATACCACTCAACCCATGGTACCC
GTGCCTCAGGGGTGAGCTGGTTGTTGCCTTGCGGTAGAGTAATAACGATAGCTCAGCCTTGCAGGTGATTTCCGCGTCTGTC
TATTGTCCTTATTACTGTGTCGAGTCCCCAAGTTTTCTTCCAATAGACATCA
SEQ5MamdSfw
SEQ ID NO: 27
GTTCTGCCTTGGGTTTAGGATGTACGACGTATATCC
SEQ5MamdSrv
SEQ ID NO: 28
GTCTTTCTCTCGTTTATGATGTCTATTGGAAGAAAACTTGG
SEQ5MKO1fw
SEQ ID NO: 29
ACTCTCTATCTGCATCAAC
SEQ5MKO1rv
SEQ ID NO: 30
GATCCCCGATTTCTTTGG
SEQ5MKO2fw
SEQ ID NO: 31
TGATGTGCTTGATATTGGGC
SEQ5MKO2rv
SEQ ID NO: 32
CTCCATCGCTCAACTATGTG

1. Process for production of a whole broth enzyme composition, comprising the following steps:
(a) providing a fermentation medium, originating from hydrolysis of lignocellulosic biomass, with a glucose content of from 5 to 450 g/L, a xylose content of from 2 to 300 g/L, a density of from 1 to 2 kg/L and a dry matter content of from 10 to 75 wt.-%;
(b) adding at least one filamentous fungus cell wherein SEQ ID NO:1 has been disrupted;
(c) mixing the fermentation medium and the at least one filamentous fungus cell for a time period of from 1 minute to 10 days at a temperature of from 20 to 35° C.; and
(d) obtaining a whole broth enzyme composition;
wherein the filamentous fungus cell is selected from the species Trichoderma reesei.

2. Process according to claim 1, wherein the pH of the fermentation medium according to step (a) has been adjusted to a pH selected from pH 2.0 to 6.0.

3. Process according to claim 1, wherein the ratio of said glucose content to said xylose content is from 1.0 to 3.5.

4. Process according to claim 1, further comprising the step
(ai) concentrating the fermentation medium by evaporation, membrane filtration or thin layer evaporation to decrease the weight of the fermentation medium by a factor of 2 to 6.

5. Process according to claim 4, further comprising the step
(aii) sterilizing the fermentation medium according to step (a) or the concentrated fermentation medium according to step (ai).

6. Process according to claim 1, wherein the fermentation medium according to step (a) has a furfural content of less than 0.5 g/L.

7. Process according to claim 1, wherein the fermentation medium according to step (a) has a hydroxymethyl furfural (HMF) content of less than 0.5 g/L.

8. Process according to claim 1, further comprising the step
(e) performing a solid-liquid separation of the fermented medium obtained from step (c) to obtain a solid fraction and a liquid fraction.

9. Process according to claim 1, wherein from 0.05 to 5 wt.-% nitrogen is added during step (a) and/or (b) of the process.

10. Process according to claim 1, wherein from 0.5 to 350-mg/L FeSO₄, MnSO₄, MgSO₄ and/or ZnSO₄ are added during step (a) and/or (b) of the process.

11. Process according to claim 1, wherein the filamentous fungus cell comprises at least one heterologous beta-glucosidase enzyme.

12. Process according to claim 1, wherein SEQ ID NO: 11 also has been disrupted in said at least one filamentous fungus cell.

13. Filamentous fungus cell wherein SEQ ID NO:1 has been disrupted, wherein the filamentous fungus cell is selected from the species Trichoderma reesei.

14. Filamentous fungus cell according to claim 13, wherein SEQ ID NO: 11 also has been disrupted.

15. Filamentous fungus cell according to claim 14, wherein SEQ ID NO:1 and/or SEQ ID NO: 11 have been disrupted by deletion, mutation, modification of a promotor or any other regulatory sequence, generation of a stop codon or RNA interference.

16. Filamentous fungus cell according to claim 13, wherein the at least one filamentous fungus cell is a genetically modified filamentous fungus cell with the ability to express at least one heterologous hydrolase enzyme, at least one heterologous pectinase enzyme, at least one heterologous oxidative enzyme and/or at least one heterologous accessory protein.

17. Filamentous fungus cell according to claim 13, wherein the at least one filamentous fungus cell is a genetically modified filamentous fungus cell comprising at least one heterologous beta glucosidase enzyme encoding sequence, at least one heterologous beta-xylosidase enzyme encoding sequence, at least one heterologous xylanase enzyme encoding sequence, at least one heterologous pectinase enzyme encoding sequence, at least one heterologous lytic polysaccharide monooxygenase enzyme encoding sequence, at least one heterologous oxidative enzyme encoding sequence and/or at least one heterologous accessory protein encoding sequence.

18. (canceled)
